(19) United States
(12) Patent Application Publication
(10) Pub. No.: US 2012/0183956 A1
(43) Pub. Date: Jul. 19, 2012

(54) SAMPLE HANDLING

(75) Inventors: Gary Ross, Gwent (GB); Sebastian Wetherall, Gwent (GB); Owen Evans, Gwent (GB); Andrew Hopwood, Birmingham (GB); Nina Moran, Birmingham (GB)

(73) Assignees: Grid Xitek Limited, Gwent (GB); Forensic Science Service Limited, Birmingham (GB)

(21) Appl. No.: 13/389,569

(22) PCT Filed: Aug. 10, 2010
(86) PCT No.: PCT/GB10/51325

§ 371 Date: Apr. 3, 2012

(30) Foreign Application Priority Data
Aug. 10, 2009 (GB) 0913903.1

(51) Int. Cl. C12Q 1/68; C12M 1/00

(52) U.S. Cl. 435/6.1; 435/306.1

(57) ABSTRACT

The invention provides containers and methods of use for the storage, transportation and preparation of samples, such as DNA samples for analysis. The container is pre-provided with the reagents in sealed chambers. The sample can be introduced and the container manipulated to release the reagents, provide the necessary conditions and give a fully prepared sample. The container can then be engaged with an analysis device to identify characteristics of the sample or perform other operations thereon.

This invention concerns improvements in and relating to sample handling, particularly, but not exclusively in relation to biological sample handling in preparation for analysis.

Various approach exist for handling samples from the point of collection up until their analysis. Problems exist with such approaches during at least a part of the process because of the risk of contamination or other sub-optimal sample handling. These problems are increased in significance where attempts are made to handle samples outside of a laboratory and/or using staff who are not trained to the same level as laboratory staff. There is an increasing need to handle such samples in such ways to reduce the processing time from collection to result and to reduce the overall cost of the process.

According to a first aspect of the invention we provide a container, the container including an opening to a chamber, closure means for the opening and discharge means for the container.

According to a second aspect of the invention we provide a method of preparing a sample, the method including:

collecting the sample;

introducing the sample to a container;

one or more preparing steps being applied to the sample whilst in the container.

The first and/or second aspects of the invention may include any of the features, options or possibilities set out in this document, including the following.

The container may be a lysis container. The container may be a reagent or material storage container.

The container may be at least partially transparent. Preferably the chamber is at least partially transparent. In this way the internal operation and orientation of the container and/or its components may be observed.

The container may be provided in or be adapted to be received within a receiving location of a unit. A plurality of containers may be so provided.

The unit may provide transport and/or storage and/or processing of the one or more containers provided therein. The containers may be removed from the unit for dispensing into the device or may be dispensed into the device whilst in the unit.

The unit may be provided with at least 5 receiving location, more preferably at least 10 receiving locations. The receiving locations may be arranged in a line or in a grid.

The sample may be a sample of biological material, for instance a DNA sample. The sample may be a blood sample or bodily fluid sample or cell containing sample or hair sample or swab, for instance a buccal swab. The sample may arrive and/or be received from one or more of: a solid matrix, a solid matrix containing fibres, paper, a swab, a buccal swab, a cotton swab, a soft swab, a solution, a suspension, an item of clothing, an item placed in the mouth, a cigarette or piece thereof, chewing gum, one or more hairs, a bone sample, a tissue sample or saliva. The sample may be physically bound to a sample collection device. The sample may be chemically bound to a sample collection device. The sample may be dried onto a sample collection device. The sample collection device may include one or more chemicals and/or reagents.

The one or more preparing steps may include a lysis step. The one or more preparing steps may include a heating step. The one or more preparing steps may include an agitation step. The prepared sample may be analysed, for instance using an analysis device.

The one or more preparing steps may include the transfer of a sample from outside a container to inside a container. The container may be opened, the sample may be introduced and the container may be closed again. The transfer of a sample may include detaching a part of the sample from another part of the sample, for instance part of the matrix from another part of the matrix. The part of the sample may be detached by punching a part of the matrix out of the rest of the matrix. The part of the sample may be detached by cutting a part of the matrix off the rest of the matrix.

The one or more preparing steps may include contact with one or more chemicals and/or reagents. The chemicals and/or reagents may buffer the sample during one or more steps. The chemicals and/or reagents may alter the sample during one or more steps, for instance by lysing one or more parts of the sample. The chemicals and/or reagents may detach and/or remove and/or release the sample or a part thereof from the form in which the sample is received from the previous step, for instance from a sample collection device or part thereof.

The one or more preparing steps may be provided with one or more conditions which differ from ambient conditions. The conditions may differ in the temperature compared with ambient and/or the level of gravity compared with ambient, for instance due to heating of the sample and/or a container therefore and/or centrifuging. The conditions may alter the sample during one or more steps. The conditions may detach and/or remove and/or release the sample or a part thereof from the form in which the sample is received from the previous step, for instance from a sample collection device or part thereof.

The one or more preparing steps receive the sample attached to a solid, such as a matrix.

The one or more preparing steps may include contacting the sample with one or more reagents and/or chemicals and/or other components. The reagents and/or chemicals and/or other components may be used to prepare the sample for one or more of the subsequent steps.

The one or more preparing steps may include contacting the sample with one or more reagents and/or chemicals and/or components which select the sample component(s) relative to one or more waste components in the sample. The selected sample component(s) may be removed from the waste component(s) and/or the waste component(s) may be removed from the selected sample components.

The one or more preparing steps may include eluting at least a part of the sample, for instance from a sample collection device or part thereof.

The one or more preparing steps may include a purification step. The purification step may separate the selected sample components, for instance DNA, from one or more waste components of the sample, for instance cellular material, PCR inhibitors and chemical inhibitors.

The one or more preparing steps may include a washing step. The washing step may remove one or more components of the sample from the location of one or more other components of the sample.

The one or more preparing steps may include an elution step. The elution step may remove one or more components of the sample from a first form into a second form. The first form may be bound to a matrix or surface or substrate, for instance on a bead. The second form may be in a liquid, for instance the eluent.

One or more reagents and/or chemicals and/or other components may be introduced during one or more of the preparing steps. The container may be opened to provide these reagents and/or chemicals and/or other components. The container may be sealed after they are introduced. More preferably, the container may be pre-provided with these reagents and/or chemicals.

One or more of the one or more preparing steps may be provided during transit of the container from a first location to a second location. The first location may be the collection location for the sample, for instance a crime scene or location within a police or law enforcement authority building. The second location may be the analysis location for the sample, for instance a laboratory or a room or location separate from the location within a police or law enforcement authority building where the sample is collected. The transit include motorised transport for the container. The container may be provided in a protective packaging during transit.

The container may have a pre-use state. The container may be stored in the pre-use state. The container may include one or more reagents in the pre-use state.

The container may have a use state. One or more chambers within the container may be accessible in the use state, for instance, accessible to a sample collection device.

The container may have a sealed use state. One or more, preferably all, the chambers within the container may be inaccessible in the sealed use state, for instance, inaccessible to a sample collection device and/or inaccessible to contamination sources.

The container may have a sample to fluid contacting state. For instance, the fluid may be released into one or more chambers containing the sample.

The container may have a sample to sample processing aids contacting state. For instance, the one or more sample processing aids, such as magnetic beads, may be released into one or more chambers containing the sample or vice versa.

The container may have an initial processing state. The container and/or its contents may be heated and/or agitated in the initial processing state.

The container may have a loading state.

The container may have an initial loading state, potentially within the loading state. The container may be introduced to a device, for instance, an analysis device in the loading state and/or initial loading state.

The container may have a transfer state, potentially within the loading state. The container may transfer at least a part of the contents of the container to the device in the transfer state. A fluid flow path between the container and the device may be formed in the transfer state. Where provided on a solid matrix or the like, the solid matrix or the like may be retained in the container during transfer.

The container or at a least a part thereof may have an expanded state and a compressed state. Fluid may pass from the container to the device during the transition between expanded state to compressed state and/or whilst in the compressed state.

The container may have a post use state. The container may be stored in the post use state. The container may be discarded in the post use state. Where provided on a solid matrix or the like, the solid matrix or the like may be retained in the container in the post-use state.

The container may progress through two or more of the following states: a pre-use state; a use state; a sealed use state; a sample to fluid contacting state; a sample to one or more sample processing aids contacting state; an initial processing state; a loading state; an initial loading state; a transfer state; an expanded state; a compressed state; a post use state. The container may progress through any two or more of the listed states in the sequence listed.

The container may be provided with an internal heat source.

The container may be provided with a single chamber.

The container may be provided with a plurality of chambers. One or more chambers may be provided which are physically isolated from one or more of the other chambers, for instance when the container is in one or more states, for instance a pre-use state.

The container may be provided with one or more chambers into which the sample is introduced. The container may be provided with one or more other chambers, for instance providing one or more reagents. The one or more other chambers are preferably separated from the one or more chambers by a seal or a plurality of seals. The container may be provided with one or more further chambers, for providing one or more sample processing aids. The one or more further chambers are preferably separated from the one or more chambers and/or one or more other chambers by a seal or a plurality of seals.

The container may include means to break the seal between the one or more chambers and the one or more other chambers and/or the one or more further chambers. Relative movement of the one or more chambers compared with the one or more other chambers chambers and/or the one or more further chambers may break the seal between them. The container may be provided with a chamber in to which the sample is introduced and another chamber into which the reagents are introduced or are present within the chamber and/or a further chamber into which one or more sample processing aids are introduced or are present within the further chamber.

The one or more other chambers may be less than 50%, more preferably less than 30% and ideally less than 10% of the volume of the chamber. The one or more other chambers may be axially spaced from the chamber. The one or more other chambers may be within a projection of the cross-section of the chamber considered along the axis or centre-line of the chamber. A other chamber may be defined by one or more walls in combination with a seal.

The seal may separate the other chamber from one or more other chambers and/or one or more chamber sand/or one or more further chambers. The one or more other chambers may be off set from the axis of the container, preferably the long axis thereof. The one or more other chambers may be provided adjacent a wall of the container. The one or more other chambers may be provided by a base section of the container, for instance a base section of or connected to a first component and/or a side portion of a first component. The one or more other chambers may be provided in a further component, for instance in the base of the further component.

The one or more further chambers may be less than 50%, more preferably less than 30% and ideally less than 10% of the volume of the chamber. The one or more further chambers may be axially spaced from the chamber. The one or more further chambers may be within a projection of the cross-section of the chamber considered along the axis or centre-line of the chamber. A further chamber may be defined by one or more walls in combination with a seal. The seal may separate the further chamber from one or more further chambers and/or one or more chambers and/or one or more other chambers. The one or more further chambers may be off set from the axis of the container, preferably the long axis thereof. The one or more further chambers may be provided adjacent a wall of the container. The one or more further chambers may be provided by a base section of the container, for instance a base section of or connected to a first component and/or a side portion of a first component. The one or more further chambers may be provided in a further component, for instance in the base of the further component.

The one or more other chambers and/or one or more further chambers may be provided above the chamber in one or more of the states, for instance in one or more of the pre-use state; a use state; a sealed use state; a sample to fluid contacting state; a sample to one or more sample processing aids contacting state; an initial processing state; a loading state; an initial loading state; a transfer state; an expanded state; a compressed state; a post use state.

The container may be provided with a first chamber which is accessed through the opening. The container may be provided with a second chamber which is accessed through the first chamber, particularly the base of the first chamber. The first and/or second chamber may be provided with one or more further chambers and/or one or more other chambers, including those of the type mentioned elsewhere within this document. The one or more further chambers and/or one or more other chambers may particularly be provided leading into the second chamber and/or in the base of the first chamber.

The container may be provided with a pre-first chamber through which the first chamber is accessed, particularly when introducing the sample.

The one or more other chambers may be provided on the side of the container, for instance on the side of a first chamber defining section. The one or more other chambers may be less than 50%, more preferably less than 30% and ideally less than 10% of the volume of the chamber. A other chamber may be defined by one or more walls in combination with a seal. The seal may separate the other chamber from one or more other chambers and/or one or more chamber sand/or one or more further chambers. The one or more other chambers may be provided as deformable components. The deformable components may be deformed by the application of external pressure, such as a finger or fingers of a user.

The one or more further chambers may be provided on the side of the container, for instance on the side of a first chamber defining section. The one or more further chambers may be less than 50%, more preferably less than 30% and ideally less than 10% of the volume of the chamber. A further chamber may be defined by one or more walls in combination with a seal. The seal may separate the further chamber from one or more other chambers and/or one or more chambers and/or one or more further chambers. The one or more further chambers may be provided as deformable components. The deformable components may be deformed by the application of external pressure, such as a finger or fingers of a user.

The one or more other chambers may be positioned intermediate the central axis of the container and the perimeter thereof. One or more other chambers which are parts of an annulus may be provided. The one or more other chambers may be provided around an opening through which the sample is introduced. The one or more another chambers may be provide at the junction between a pre-first chamber and a first chamber.

The container may have a consistent cross-sectional area along an axis perpendicular to its cross-section. A circular cross-section may be provided.

The container may be have an irregular hexagonal cross-section. The length of one or two pairs of side lengths may be the same. The length may be less than the length of one or two pairs of other side lengths.

The container may be provided with a lid. The lid may have a circular cross-section. The lid may have a cross-section to enable at least a part of it to be received in a first chamber or a pre-first chamber. The lid may have a cross-section to enable at least a part of it to be received around the perimeter of a first chamber or a pre-first chamber.

The container may include one or more side walls and one or two end walls.

The container may have a circular cross-section. The container may be a right cylinder, for instance with a lid.

The single chamber may be defined by a plurality of components of the container. The components may be or include a pair of coaxial cylindrical components. The radius of one cylindrical component may be less than the radius of the other. One of the plurality of components may be slidably received within another. One of the plurality of components may provide a plunger, for instance for dispensing fluid from the container.

The container may include a first component in which another component is slidably received. The first component may provide one chamber for the container, the another component may provide another chamber for the container. The another component may include a passageway to accommodate on or more parts of the first component, for instance one or more swab retaining elements or structures. The passageway may be centrally provided. The passageway may be circular in cross-section. The first component and/or another component may be cylindrical. The another component may be cylindrical, with a central cylindrical passageway and annular end walls. One or more sealed opening may be provided between the one or more chambers and one or more another chambers.

The container, particularly the chamber thereof, may be provided with one or more structures and/or components to promote heat transfer from the outside of the container to the inside thereof. Heat transfer from the outside of the chamber to the inside of the chamber may particularly be provided.

The container may be provided with one or more temperature monitoring devices. The temperature monitoring device may be one or more sensors in and/or on the container. The temperature monitoring device may be a characteristic change, for instance a colour change, triggered by a temperature or temperatures. The change may be reversible.

The container may include a first section which is rotatable relative to a second section.

The first section may be an outer lid. The second section may be an inner lid.

The first section may be a base section. The second section may be the container and/or a chamber defining section.

The first section may be a first chamber defining section. The second section may be a second chamber defining section.

The first section may be a pre-first chamber defining section. The second section may be a first chamber defining section.

The container may include an outer lid and an inner lid. The outer lid may be rotatable relative to the inner lid. The outer lid may be rotatable about the container and/or the inner lid may be rotatable. The outer lid may be provided with one or more openings. The inner lid may be provided with one or more openings. One or more or all of the openings on the outer lid and/or inner lid may be provided with seals and/or valve elements and/or closures. One or more of the openings may be configured with a cross-section corresponding to the cross-section, perpendicular to its direction of insertion, of the sample collection device, such as a swab.

In the use state, an opening in the outer lid may be aligned with an opening in the inner lid. The sample, for instance on a swab, may be introduced through the aligned openings.

In the sealed use state, the opening in the outer lid which was aligned with the opening in the inner lid in the use state may be moved out of alignment. In the sealed use state no opening on the outer lid may be aligned with an opening in the inner lid, preferably even partially.

One or more stops or other locator means may be provided to control the position of the inner lid and outer lid relative to one another and/or relative to the container.

In the pre-use state and/or use state and/or sealed use state and/or initial processing state and/or expanded state and/or compressed state and/or initial loading state and/or post use state, the flow path forming element, for instance a needle, provided on the container may not be aligned with an opening in the outer lid.

In the transfer state, the flow path forming element, for instance a needle, provided on the container may be aligned with an opening in the outer lid. The opening may be provided with a seal. The outer lid and/or inner lid and/or container may be rotated to align the flow path forming element with the opening.

The container may include a base section, preferably mounted on the container. The base section may be rotatable relative to the container. The container, particularly the base thereof, may be provided with one or more openings. The base section may be provided with one or more holding locations. The base section may be provided with one or more openings for each holding location. One or more or all of the openings in the base of the container and/or in the base section may be provided with seals and/or valve elements and/or closures.

In the use state, an opening in the base section may be aligned with an opening in the container. One or more materials, such as a reagent(s), may enter the chamber through the aligned opening(s).

One or more stops or other locator means may be provided to control the position of the base section and container relative to one another.

The container may include a first chamber defining section and a second chamber defining section. The first chamber defining section may be at least partially received within the second chamber defining section. The first chamber defining section may also provide the pre-first chamber defining section. The first chamber defining section may be rotatable relative to the second chamber defining section.

One or more stops or other locator means may be provided to control the position of the first chamber defining section relative to the second chamber defining section, for instance by means of a locator means being confined by a track. The locator means may be provided on the first chamber defining section and the track may be defined on the second chamber defining section.

The track may include a first perimeter extending, ideally circumferentially extending, section. The track may include one or more further perimeter extending, ideally circumferentially extending, sections. The track may include one or more axially extending sections. One perimeter extending section may be joined to another by an axially extending section.

In the pre-use state and/or use state and/or sealed use state, a protrusion in the container may not be aligned with an opening in the container. Preferably the protrusion is provided on the second chamber defining section and/or in the second chamber. Preferably the opening is provided on the first chamber defining section.

In the initial processing state and/or expanded state and/or compressed state and/or initial loading state and/or post use state and/or transfer state, the protrusion may be aligned with an opening in the container. The alignment may be provided by rotation, for instance rotation along a first perimeter section.

In the initial processing state and/or expanded state and/or compressed state and/or initial loading state and/or post use state and/or transfer state, the protrusion may be at least partially within one or more other chambers and/or further chambers and/or holding locations. The protrusion may be so provided by movement, for instance along the axial section of the track.

In the initial processing state and/or expanded state and/or compressed state and/or initial loading state and/or post use state and/or transfer state, the protrusion may be aligned with an opening in the container, preferably at least partially within one or more other chambers and/or further chambers and/or holding locations, but in a different alignment to the initial alignment provided. The protrusion may be so provided by rotation, for instance along the further perimeter section of the track.

The container may include a pre-first chamber defining section and a first chamber and/or second chamber defining section. The pre-first chamber defining section may be at least partially received within the first chamber defining section. The pre-first chamber defining section may be rotatable relative to the first and/or second chamber defining section.

One or more stops or other locator means may be provided to control the position of the pre-first chamber defining section relative to the first and/or second chamber defining section, for instance by means of a locator means being confined by a track. The locator means may be provided on the pre-first chamber defining section and the track may be defined on the first and/or second chamber defining section or vice versa.

The track may include a first perimeter extending, ideally circumferentially extending, section. The track may include one or more further perimeter extending, ideally circumferentially extending, sections. The track may include one or more axially extending sections. One perimeter extending section may be joined to another by an axially extending section.

In the pre-use state and/or use state and/or sealed use state, a protrusion in the container may not be aligned with an opening in the container. Preferably the protrusion is provided on the second chamber defining section and/or in the second chamber. Preferably the opening is provided on the first chamber defining section.

In the initial processing state and/or expanded state and/or compressed state and/or initial loading state and/or post use state and/or transfer state, the protrusion may be aligned with an opening in the container. The alignment may be provided by rotation, for instance rotation along a first perimeter section.

In the initial processing state and/or expanded state and/or compressed state and/or initial loading state and/or post use state and/or transfer state, the protrusion may be at least partially within one or more other chambers and/or further chambers and/or holding locations. The protrusion may be so provided by movement, for instance along the axial section of the track.

In the initial processing state and/or expanded state and/or compressed state and/or initial loading state and/or post use state and/or transfer state, the protrusion may be aligned with an opening in the container, preferably at least partially within one or more other chambers and/or further chambers and/or holding locations, but in a different alignment to the initial alignment provided. The protrusion may be so provided by rotation, for instance along the further perimeter section of the track.

The container may contain one or more reagents or materials in the pre-use state, for instance one or more lysis reagents, preferably in fluid form.

The container may contain one or more sample processing aids. The one or more sample processing aids may provide their function in the container. The one or more sample processing aids may provide their function after dispensing from the container, for instance in a process unit to which at least a part of the sample is introduced. The one or more sample processing aids may be liquid and/or solid. The one or more sample processing aids may include a magnetic element, for instance as beads, ideally as beads with a magnetic core. The one or more sample processing aids may be used to assist and/or may be used to provide a separation between one or more components of the sample and one or more other components. The separation may include the separation of DNA from one or more other components. The separation may be a partial or complete separation.

The container may contain one or more reagents or material for amplification, preferably PCR based amplification. The one or more reagents or material for amplification may provide their function in the container. The one or more reagents or material for amplification may provide their function after dispensing from the container, for instance in a process unit to which at least a part of the sample is introduced. The one or more reagents or material for amplification may be liquid and/or solid.

The container may contain one or more reagents or materials for use in a size based separation and/or for use in electrophoresis, such as capillary electrophoresis. The one or more reagents may be the material in which the separation and/or electrophoresis is performed. The one or more reagents or materials for use in a size based separation and/or for use in electrophoresis may provide their function in the container. The one or more reagents or materials for use in a size based separation and/or for use in electrophoresis may provide their function after dispensing from the container, for instance in a process unit to which at least a part of the sample is introduced. The one or more reagents or materials for use in a size based separation and/or for use in electrophoresis may be liquid and/or solid.

The container may contain one or more of the components used in the processing of the sample, for instance, one or more electrochemical pump feed materials or one or more buffers or one or more liquids used to move or displace the sample within the process. The one or more of the components used in the processing of the sample may provide their function in the container. The one or more of the components used in the processing of the sample may provide their function after dispensing from the container, for instance in a process unit to which at least a part of the sample is introduced. The one or more of the components used in the processing of the sample may be liquid and/or solid.

A set of containers may be provided, for instance in a common package, the set of containers including one or more of:

a container containing one or more reagents or materials, such as lysis reagents or materials;

a container containing one or more sample processing aids, such as magnetic beads;

a container adapted to receive a sample;

a container containing one or more reagents or material for amplification, preferably PCR based amplification;

a container containing one or more reagents or materials for use in a size based separation and/or for use in electrophoresis, such as capillary electrophoresis, for instance the material in which the separation and/or electrophoresis is performed;

a container containing one or more of the components used in the processing of the sample, for instance, one or more electrochemical pump feed materials or one or more buffers or one or more liquids used to move or displace the sample within the process.

The container may be provided with a chamber for one or more reagents and a separate chamber for one or more other reagents and/or one or more sample processing aids. The separation between the chamber and the separate chamber may be at least partially removed during a stage for the container.

The container may be provided with a chamber for one or more reagents and a separate chamber for one or more other reagents and a further separate chamber for one or more sample processing aids. The separation between the chamber and the separate chamber and/or the chamber and the further separate chamber and/or the separate chamber and the further separate chamber and/or between all three chambers may be at least partially removed during a stage for the container.

The separation between the chamber and the separate chamber and/or the chamber and the further separate chamber and/or the separate chamber and the further separate chamber and/or between all three chambers may be at least partially removed during two or more separate stages for the container. For instance, the separation between the chamber and the separate chamber may be at least partially removed, followed at a later point in time by the at least partial removal of the separation between the separate chamber or chamber and the further separate chamber.

The chamber, separate chamber and further chamber may be provided with a common axis. One or two of the chamber, separate chamber and further chamber may be received in one or more stages within one or two of the other of the chamber, separate chamber and further chamber, for instance by means of a sliding reception of one in the other. The chamber, separate chamber and further chamber may be provided with corresponding shaped, but different size profiles to one another.

The chamber, separate chamber and further chamber may be provided together with one or more other chambers.

The container may be provided with an opening, for instance in an end wall of the container. A single opening may be provided.

The opening may be provided in the base of the container. The opening alternatively or additionally may be provided in a base section, for instance mounted on the container.

The opening may be provided in a first chamber of the container or a pre-first chamber of the container.

The opening may be provided with the closure means, for instance by sealing. The opening may be sealed by a seal and/or a valve element. The opening may be sealed in the pre-use state. The seal may be a foil seal. The seal may be removed by peeling the seal from the container. The seal may be a lid.

The lid may be provided with a screw-thread type engagement with the container. The lid may be removed to provide the container in the use state.

The inside of the container may be physically isolated from the outside of the container and/or protected against contamination arising from outside the container, particularly in the pre-use state.

The container may be moved from a pre-use state to a use state by breaking the seal on the opening to the container. The seal may be broken by removing a part or the whole of the seal.

The seal may be broken by piercing a part or the whole of the seal. The seal may be broken may deforming a part or the whole of the seal.

The seal may be broke by removing the lid that provides the seal.

An opening in the container may be provided with a valve element. The valve element may be in the form of a flexible diaphragm or seal, for instance a rubber diaphragm or seal. The valve element may include an opening. The valve element may include a slit. The valve element may bias the edges of the opening and/or slit together or into overlap.

The valve element may be provided between a pre-first chamber and a first chamber provided in the container.

The valve element may have a first valve state, preferably in which the valve resists the passage of material, particularly fluids, through the valve element. The valve element may have a second valve state, preferably in which the valve allows the passage of material, particularly an element, through the valve element.

Preferably the valve element is physically isolated from the outside of the container and/or its environments in the pre-use state, for instance by a seal. The valve element may be exposed in the use state. The valve element may be exposed by removing a seal. Preferably the valve element is physically isolated from the outside of the container and/or its environments in one or more of: a sealed use state; an initial processing state; a loading state; an initial loading state; a transfer state; an expanded state; a compressed state.

The valve element may moved from the first valve state to the second valve state by an element. The element may be an elongate element. The element may be provided with one or more detachable parts. The element may be a sample collection device. The element may be provided with a swab part, ideally a detachable swab part. The element may be provided with an absorbent part and/or material collecting part.

The valve element may be moved from the first valve state towards the second valve state by pushing the element against the valve element. The element may cause the valve element to deform. The element may open the valve element. The element may pass through the valve element into the container. The element may break or rupture the valve element. In particular, the distal end of the element may be pushed against the valve element, for instance to break the valve element. The end of the element inside the container, particularly the detachable part and/or swab part and/or absorbent part and/or material part, may be placed in a fluid in the container.

The length, preferably the axial length, of the chamber which receives the element may be longer than the axial length of that part of the element inserted into it. The element may extend from the valve element into the chamber by a distance which is less than the distance separating the valve element and a seal provided between the chamber and the outlet from the container. For instance, the element may extend from the valve element into the chamber by a distance which is less than the distance separating the valve element and a second valve element.

The chamber provided between the valve element and the outlet from the container and/or between the valve element and a second valve element may had a width and/or diameter which is less than the width and/or diameter of the container before the valve element is reached.

The valve element may be returned from the second valve state to the first valve state by the removal of the element. The element may be pulled out of the container. The element may be discarded.

The detachable part(s) may detach from the element during the withdrawal of the element from the container. A part of the detachable part(s) may abut a part of the container, particularly a part of the valve element, during withdrawal of the element from the container.

A part of the detachable part(s) may be restrained, particularly by a part of the valve element, during withdrawal of the element from the container. The detachable part(s) may be retained in the container after removal of the element. The detachable part(s) may be retained in a first chamber of the container. The detachable part(s) may be provided in the liquid in the container.

The container may be opened to allow an element to be introduced. The element may be or may be a part of a matrix and/or sample collection device. A part may be detached from the element, particularly when in the form of a sample collection device, for isntance by cutting and/or punching. The container may be closed once the element has been introduced. The element may be introduced to a first chamber or to a pre-first chamber.

The container may include one or more swab retaining elements or structures. One or more of the swab retaining elements may be in the form of a projection. One or more of the swab retaining elements or structures may extend from an end wall, such as the base, of the container. One or more of the swab retaining elements may extend from the side of the container.

A structure may be formed by a plurality of projections. The swab retaining structure may be in the form of a group, such as a ring, of swab retaining elements.

One or more of the swab retaining elements and/or structure may have a first position and a second position. The insertion of a swab may cause the transition from the first position to or towards the second position. The removal of the swab may be resisted and/or prevented by one or more of the swab retaining elements and/or structures in the second position. One or more of the swab retaining elements may be spaced from one or more of the other swab retaining elements, for instance by a gap.

The container may be provided with a lid, particularly a lid suitable for closing an opening or the opening in the container. The lid may be separate from the opening, particularly in the pre-use state. The lid may be connected to the container, for instance by a breakable element, particularly in the pre-use state.

The lid may be inserted at least partially into the container, particularly the first chamber or pre-first chamber thereof.

The lid may be provided with one or more seals. A seal, for instance a first lid seal, may be provided across the mouth of the lid and/or to separate the inside of the lid from the outside of the lid. A seal, for instance a second lid seal, may be provided on the outer surface of the lid, for instance in opposition to the first lid seal.

The lid may be separated from the container, for instance by breaking the element connecting the lid to the container. The seal, particularly the first lid seal, may be removed prior to the lid being placed over the opening.

The perimeter of the opening, or at least a part thereof, may abut a deformable element in the sealed use position. The deformable element may be a cap, particularly a rubber cap, provided within the lid. The deformable element is preferable free from any slits or other apertures.

The second lid seal may be provided on the outside surface of the deformable element.

The same lid may close the opening in the pre-use state and in the sealed use state.

The container may change from the use state to the sealed use state by the application of a lid to the opening into the container.

The initial processing state may be applied by a device. The device may be the analysis device, for instance by virtue of a part of the device. The device may be separate from the analysis device.

The container may be provided with a loading state after one or more of the use state, the sealed use state, the initial processing state. The loading state may include an initial loading state and a transfer state subsequent thereto.

The container may be provided with discharge means in the form of an outlet opening. The outlet opening may be the same opening as the opening.

The container may be provided with an outlet opening in an end wall of the container, for instance the top of the container.

The outlet opening may be provided with one or more seals and/or valve elements and/or a lid. A seal may be provided to seal access to the inside of the container relative to the outside of the container and/or the environment. The seal may be provided on a lid for the container. The seal may be removable.

The outlet opening may be provided elsewhere on the container, for instance in the end of the container opposite the opening, for instance through which the swab is introduced. The outlet opening may be provided in an end wall of the container, for instance the base of the container.

The container may be provided with an outlet in the base of the container. The container may be provided with an outlet in the second chamber, for instance the base thereof.

The base of one or more chambers, particularly the first chamber, may be inclined down towards the opening. The seal may be provided at the bottom of the opening.

The outlet opening may be recessed relative to one or more adjacent parts of the container. The outlet opening may be recessed relative to a perimeter which defines a base of the container. The seal for the outlet opening may be similarly provided. The outlet opening and/or seal therefor may be separated from a plane defined by the base of the container.

The container and/or a portion thereof may be configured to cooperate with a location, for instance an inlet location, on a device. The device may be the analysis device for the sample.

The container may be have an external profile which at least in part corresponds to the internal profile of an inlet location on the device. The cross-sections of the container and inlet perpendicular to the direction of insertion of the container into the inlet location may in part or wholly correspond.

The container may have an external screw thread which cooperates with an internal screw thread on the device or vice versa. A bayonet style engagement between the container and the device may be provided. A luer style engagement may be provided between the container and the device.

The outlet opening seal for the container may be removed and/or be broken as part of the loading state, particularly the initial loading state.

The outlet opening on the container may adjoin, and preferably abut, a part of the inlet location on the device. The outlet opening on the container, for instance the lid, may abut the inlet location on the device.

The movement of the container into the inlet location may cause the outlet opening and/or a component thereof, for instance the outlet opening seal, to deform and/or be pierced.

The container and/or device and/or inlet location may be provided with a piercing element. The piercing element may be a needle. The needle may break the seal for the container in the transfer state.

The loading state, particularly the initial loading state, may include the removal of the removable seal. The loading state, preferably the initial loading stage, may provide for the formation of a fluid path from the inside of the container to the inside of the inlet location and/or device.

The length of the container, preferably considered along the direction of introduction of the container to the inlet location of the device, is preferably greater than the length of the inlet location. Preferably, with a part of the container abutting a part of the device and/or inlet location of the device, further movement of the container into the device and/or inlet location is prevented. Preferably, with a part of the container abutting a part of the device and/or inlet location of the device, a section of the container projects from the inlet location and/or device.

Preferably one of the plurality of components forming the container projects from the inlet location and/or device, ideally the smaller cross-sectional area component. Preferably one of the plurality of components forming the container is received within the inlet location and/or device, ideally the larger cross-sectional area component.

A section, particularly a projecting section, of the container may be provided with a first expanded state and a second compressed state. The volume of the section and hence of the container is preferably less with the section in the second compressed state.

The section may be provided in the first expanded state during the pre-use state and/or use-state and/or sealed use state and/or initial process state and/or loading state and/or initial loading state. The section may be provided in the second compressed state during the transfer state and/or post use state.

The section may be in the form of a plunger.

The section may be the first chamber defining section and/or pre-first chamber defining section.

The section may be the pre-first chamber defining section.

The section may include a location and a second location, with the separation of the location from the second location being less in the compressed state than in the expanded state. The section may include or be of a concertina type profile.

The section may include a part which can be deformed. The deformable part may be or include a dome section, preferably with its apex away from the remainder of the container. The shape of the part may be inverted during deformation. The direction of the part, for instance the dome, may be inverted during deformation. The part may be held in the deformed position and/or compressed state.

The section may undergo a transition from the expanded to the compressed state due to the application of force, for instance to an end surface of the container. The transition from expanded to compressed state may force the fluid in the container into the inlet location and/or into the device and/or out of the container.

The section may be held in the compressed state, once reached. The section may be held so as to prevent its reversing to the expanded state. The section may be held due to the interaction of one or more components on the container with one another and/or interaction between one or more components on the container with one or more components on the device.

The section may include a moveable component, for instance a plunger, so as to reduce the volume of the section and/or provide the transition from first expanded state to second compressed state. The section may be cylindrical with a circular plunger moving axially therein. The transition between expanded and compressed state may be provided before the initial processing state.

The section may be provided by a component of the container, particularly the another component which is slidably received in the component.

The section may be restrained from moving from the first expanded state to the second compressed state. One or more restraining elements may be provided. The restraining element(s) may be one or more resilient bands. The restraining element(s) may be provided between a first part of the section and a second part of the section and/or container. The first and second parts may need to be closer together for the second compressed state to be obtained.

The one or more restraining elements may be wrapped around at least a part of the section. The one or more restraining elements may be removable. Once removed, the transition from the first expanded to the second compressed state may be possible.

The one or more restraining elements may extend from a part of the section, for instance an end of the section, across the section to another part of the section and/or of the container. The one or more restraining elements may be connected to a part of the section and/or of the container, particularly the end of the section. The one or more retraining elements may have a first restraining state and a second released state. In the first restraining state the restraining elements may cooperate with one or more parts of the container, for instance one or more protrusions thereon. In the second released state, the restraining elements may be disengaged from the one or more parts of the containers, for instance one or more protrusions. The transition from the expanded to the compressed state may be possible for the section with the restraining elements in the second state. The transition from the expanded to the compressed state may not be possible for the section with the restraining elements in the first state. The one or more restraining elements may extend down one or more sides of the container.

The transition from the first state to the second state for the restraining elements may be caused by movement of one or more components of the container relative to one or more other components of the container.

The transition from the first state to the second state for the restraining elements may be caused by insertion of the container into the inlet location and/or device. The transition may be caused by one or more components of the inlet location and/or device acting on the one or more restraining elements, for instance by displacing the restraining elements sideways and/or away from the container and/or away from the one or more parts of the container with which they were cooperating.

The transition from the first state to the second state for the restraining elements may be caused by rotation of one or more components of the container relative to one or more other components. Rotation of the one or more other components may be restrained by the inlet location and/or device. The transition from the first state to the second state for the restraining elements may be caused by movement of the restraining elements from a position where movement of the retraining elements relative to one or more components of the container is prevented by an abutment surface and/or movement of the restraining elements to a position where movement of the restraining elements relative to one or more components of the container is possible.

The container may be transferred from the use state and/or the sealed use state to the initial processing state manually, but is preferably transferred automatically.

The container may be transferred from the use state and/or the sealed use state and/or initial processing state to the loading state manually, but is preferably transferred automatically.

The container may be introduced to a processing unit. The processing unit may be separate to the device. The processing unit may provide agitation and/or heating for the container and/or the contents of the container.

The processing unit may be hand held.

The processing unit may be elongate.

The processing unit may have an internal cavity. The container may be introduced into the internal cavity. The processing unit may have an openable door for the cavity. The cavity may be profiled to correspond to one or more parts of the external profile of the container.

The container may be introduced to a processing unit which is incorporated into the device, for instance by being in the same housing as the device. The processing unit may provide agitation and/or heating for the container and/or the contents of the container.

The processing unit may be a recess in a surface of the device, for instance a right-cylindrical recess. Heat may be applied to and/or removed from one or more surfaces of the processing unit, particularly surface(s) of the recess.

The processing unit may be provided with a switch, for instance to start and/or stop processing. A multi-step switch may be provided. The multi-step switch may require a first operation and a second operation to start and/or stop processing. The multi-step switch may require a rotation of a part, followed by pressing of a part or vice versa. The parts may be the same or different parts.

The processing unit may be configured to cooperate with a location, for instance an inlet location, on a device. The device may be the analysis device for the sample.

The processing unit may be have an external profile which at least in part corresponds to the internal profile of an inlet location on the device. The cross-section or cross-sections of the processing unit and inlet perpendicular to the direction of insertion of the processing unit into the inlet location may in part or wholly correspond.

The outlet opening on the processing unit may adjoin, and preferably abut, a part of the inlet location on the device. The outlet opening on the processing unit may abut the inlet location on the device.

A component of the processing unit may be inserted into the inlet location and/or device, but preferably a component, such as a needle, of the inlet location and/or device is inserted into the processing unit.

One or more operations may be applied to the processing unit to render the transition from the first expanded to the second compressed state to be possible and/or to provide the transition from the first state to the second state for the restraining elements. The one or more operations may include a rotation of a part of the processing unit.

The processing unit may receive the container, apply one or more operations to the container and/or its contents and allow the removal of the container from the device and/or the removal of at least a part of the sample from the container and/or processing unit. The one or more operations may be applied by the processing unit is response to a user request and/or activation of a switch. The one or more operations and/or container and/or container's contents may be isolated from user intervention during the one or more operations. The one or more operations may be pre-set. The one or more operations may be varied by the processing unit, for instance in response to feed back. The one or more operations may control one or more characteristics of one or more or all of the operations and/or one or more or all of the steps within one or more or all of the operations. The characteristics may be or include one or more of the following: the length of a step, the length of an operation, the temperature or temperature profile of a step, the temperature or temperature profile of an operation, the energy or intensity or duration of agitation of a step, the energy or intensity or duration of an operation, the maintenance of a seal or the separation between one or more chambers within the container, the breaking of a seal or the separation between one or more chambers within the container, a change in position or orientation of one or more components or parts of the container, provision of a flow path out of the processing unit, the ability of a user to access the container within the processing unit, the ability of a user to remove the container from the processing unit, the ability of a user to remove or dispense a part or all of the sample in the container, the contacting of one or more reagents or materials with one or more other reagents or materials or with the sample, the contacting of one or more processing aids with the sample or one or more reagents, a variation in the position or volume or orientation of the container or one or more or all the chambers provided in the container.

The inside of the container may be physically isolated from the outside of the container and/or protected against contamination arising from outside the container in one or more of: a pre-use state; a sealed use state; a sample to fluid contacting state; an initial processing state; a loading state; an initial loading state; a transfer state; an expanded state; a compressed state; a post use state.

The container may be sealed in one or more of: a pre-use state; a sealed use state; a sample to fluid contacting state; an initial processing state.

The container may be only open to the device in one or more of: a loading state; an initial loading state; a transfer state; an expanded state; a compressed state.

The container may have a pre-use state. The container may be stored in the pre-use state. The container may include one or more reagents in the pre-use state. The pre-first chamber and/or first chamber may be provided with a seal between the pre-first chamber and/or first chamber and the environment of the container. A lid, for instance a plunger may be provided attached to the container and/or removed from the pre-first chamber and/or first chamber.

The container may have a use state. One or more chambers within the container may be accessible in the use state, for instance, accessible to a sample collection device. The transition from pre-use state to use state may be provided by removing the seal and/or breaking the seal. The sample, preferably on a sample collection device may be introduced to the container in the use state and preferably to the first chamber in the use state. The sample may be introduced to a chamber, such as the first chamber, through a further seal, ideally in diaphragm or seal form.

The container may have a sealed use state. One or more, preferably all, the chambers within the container may be inaccessible in the sealed use state, for instance, inaccessible to a sample collection device and/or inaccessible to contamination sources. The transition from use state to sealed use state may be provided by withdrawing the sample collection device, or a part thereof, and allowing the seal to close. The sample, particularly on a part of the sample collection device, may be within the container, ideally within the first chamber, in the sealed use state.

The container may have a sample to fluid and/or reagent contacting state. For instance, the fluid and/or reagent may be released into one or more chambers containing the sample. The transition from sealed use state to sample to fluid and/or reagent contacting state may be provided by movement of one or more parts of the container. The movement may be rotation and/or axial movement. The movement may cause one or more seals to be removed or broken and/or alignment of one or more apertures to arise. The movement may cause the fluid and/or reagent to enter a chamber, such as the first chamber.

The container may have a sample to sample processing aids contacting state. For instance, the one or more sample processing aids, such as magnetic beads, may be released into one or more chambers containing the sample or vice versa. The transition to sample processing aids contacting state may be provided by movement of one or more parts of the container. The movement may be rotation and/or axial movement. The movement may cause one or more seals to be removed or broken and/or alignment of one or more apertures to arise. The movement may cause the sample processing aids to enter a chamber, such as the first chamber.

The container may have an initial processing state. The container and/or its contents may be heated and/or agitated in the initial processing state. The transition to the initial processing state may be provided by inserting the container into a device and/or by activating a device.

The container may have a loading state. The transition to the loading state may be provided by connecting the container to the device.

The container may have an initial loading state, potentially within the loading state. The container may be introduced to a device, for instance, an analysis device in the loading state and/or initial loading state.

The container may have a transfer state, potentially within the loading state. The container may transfer at least a part of the contents of the container to the device in the transfer state. A fluid flow path between the container and the device may be formed in the transfer state. The transition to the transfer state may be provided by introducing the container to the device and/or by changing the position of the container on the device and/or by changing the configuration and particularly the internal volume of the container. The lid and/or plunger may be advanced into the container, for instance into the pre-first chamber and/or first chamber. The sample, or a part thereof, may be dispensed from the container into the device in the loading state.

The container or at a least a part thereof may have an expanded state and a compressed state. Fluid may pass from the container to the device during the transition between expanded state to compressed state and/or whilst in the compressed state. The transition to the compressed state may be provided by introducing the container to the device and/or by changing the position of the container on the device and/or by changing the configuration and particularly the internal volume of the container. The lid and/or plunger may be advanced into the container, for instance into the pre-first chamber and/or first chamber. The sample, or a part thereof, may be dispensed from the container into the device in the compressed state.

The container may have a post use state. The container may be stored in the post use state. The container may be discarded in the post use state.

Various embodiments of the invention will now be described, by way of example only, and with reference to the accompanying drawing, in which:

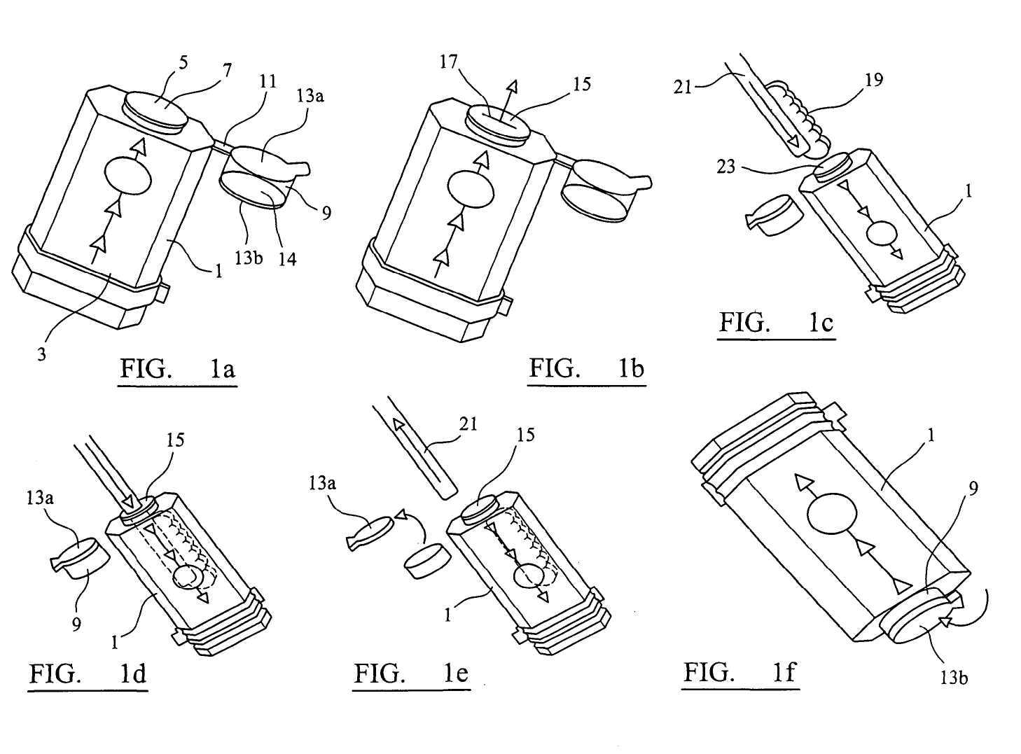

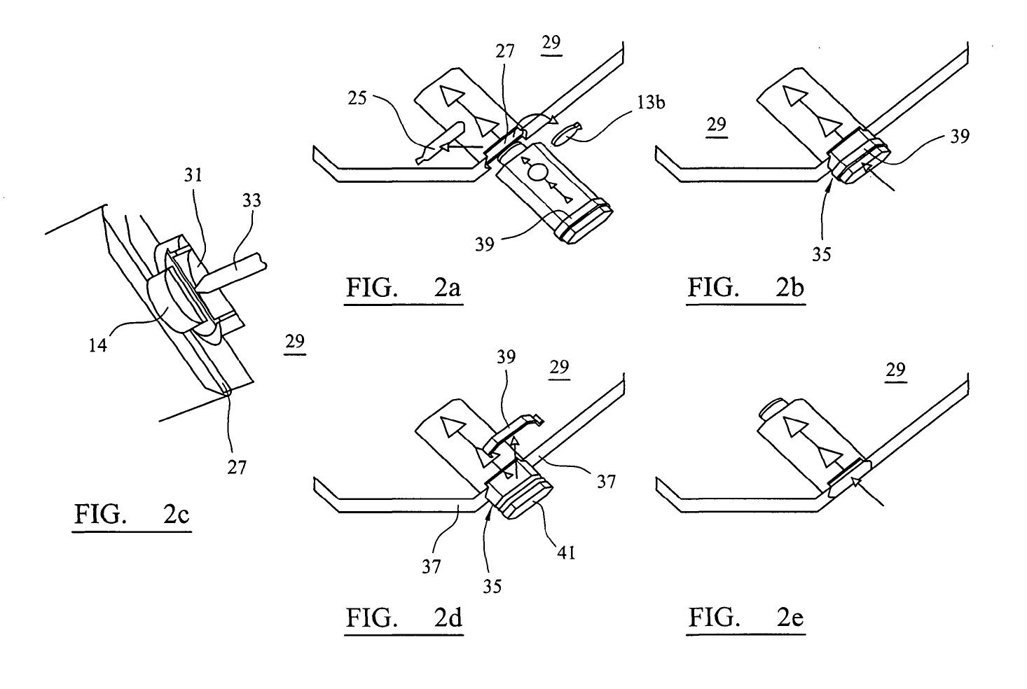

FIG. 1a shows a perspective side view of a lysis container with a lid removed;

FIG. 1b shows the lysis container of FIG. 1a with a foil seal removed;

FIG. 1c shows the lysis container of FIG. 1b with a swab applicator and swab being inserted;

FIG. 1d shows the lysis container of FIG. 1c with a swab applicator and swab fully inserted;

FIG. 1e shows the lysis container of FIG. 1d with the swab applicator removed;

FIG. 1f shows the lysis container of FIG. 1e with the lid applied;

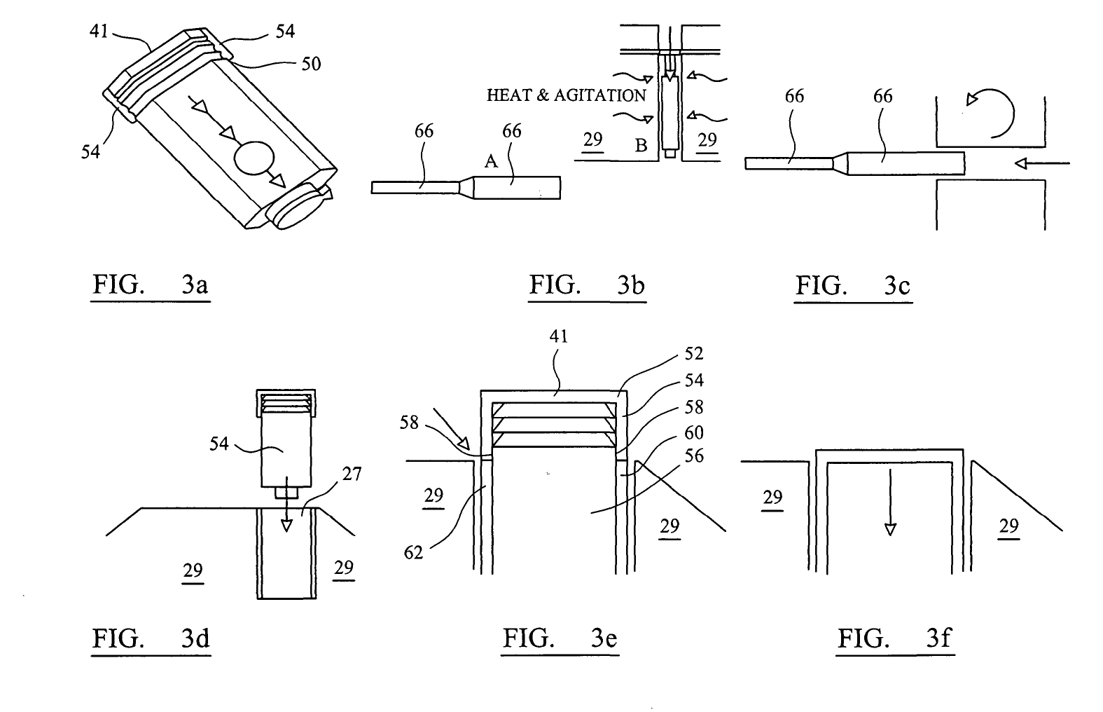

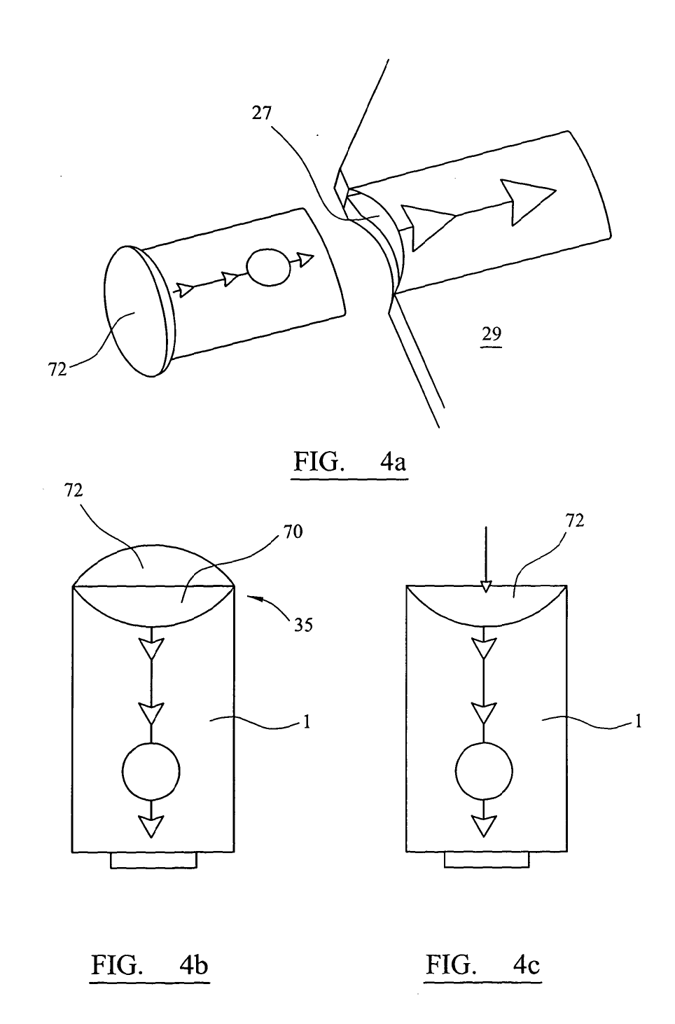

FIG. 2a shows a perspective view of the lysis container ready for insertion into a device;

FIG. 2b shows the container after insertion;

FIG. 2c shows a detailed sectional view of the container to inlet interface during insertion;

FIG. 2d shows the removal of the restraining device;

FIG. 2e shows the container after loading of the sample into the device;

FIG. 3a shows an alternative embodiment of the container;

FIG. 3b shows the container in a processing state;

FIG. 3c shows the container translating from the processing state to a loading state;

FIG. 3d shows the container being aligned with a device;

FIG. 3e shows the container and its restraining elements in a first state;

FIG. 3f shows the container and its restraining elements in a second state;

FIG. 4a shows an alternative embodiment of the container;

FIG. 4b shows the container with the section in the expanded state;

FIG. 4c shows the container with the section in the compressed state;

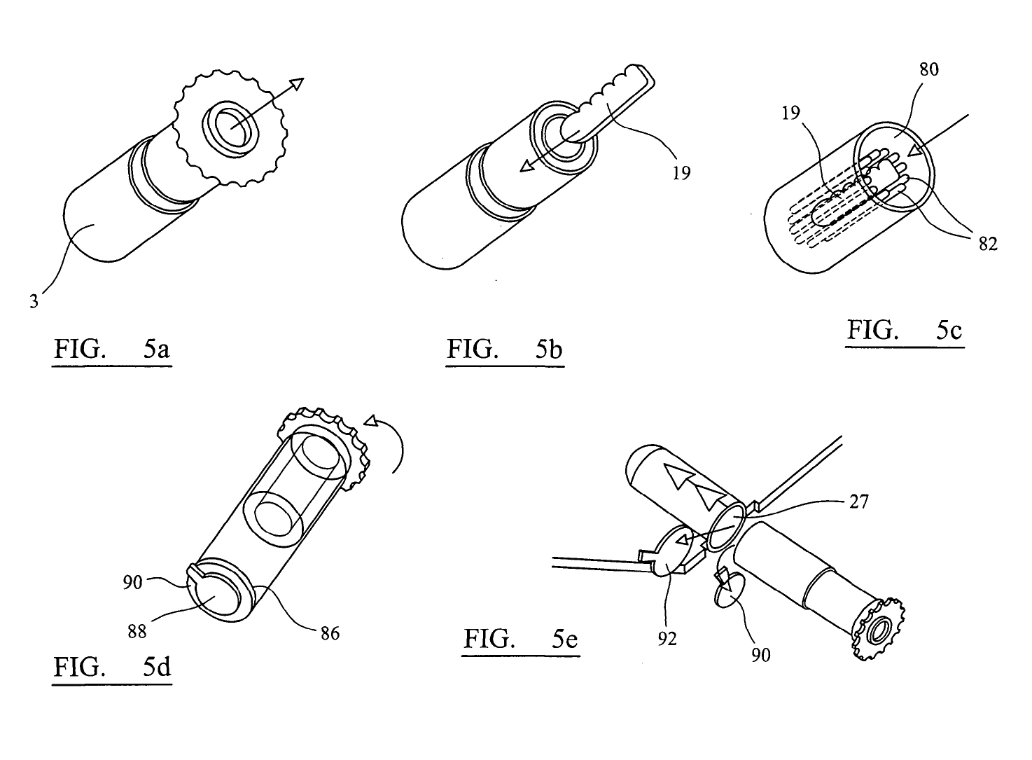

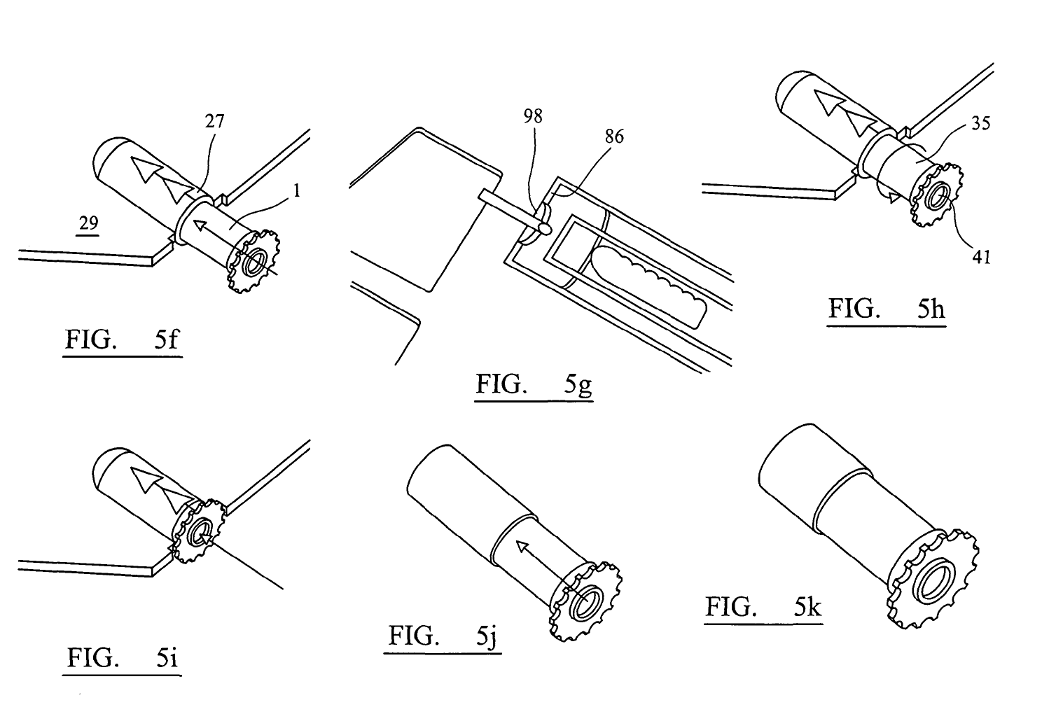

FIG. 5a shows a further embodiment of the container;

FIG. 5b shows a container FIG. 5a with the lid removed;

FIG. 5c shows the top section of the container with the swab inserted;

FIG. 5d is a perspective view of the container with the lid repositioned;

FIG. 5e is a perspective view of the container being inserted into a device;

FIG. 5f shows the container inserted into a device in the expanded state;

FIG. 5g is a perspective cross-sectional view showing the bottom part of the container inside the device;

FIG. 5h shows the container being released into the compressed state;

FIG. 5i shows the container in the compressed state;

FIG. 5j illustrates the two sections of the container in the expanded state;

FIG. 5k illustrates the two sections of the container in the compressed state;

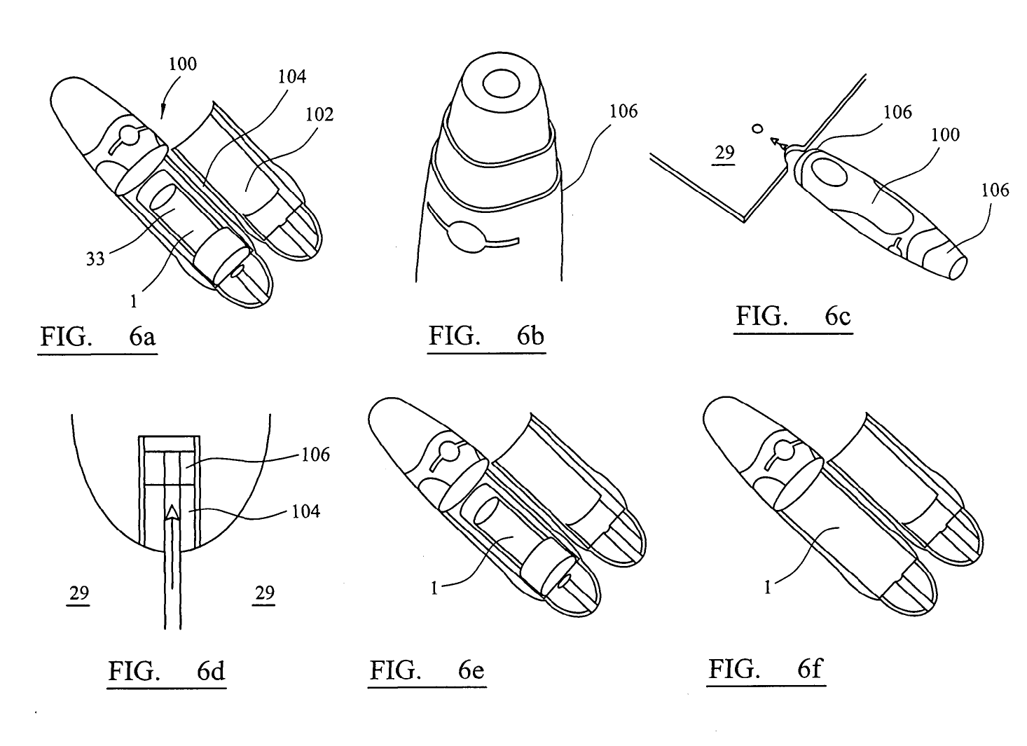

FIG. 6a shows a perspective view of a heating and agitation unit with a container in situ;

FIG. 6b shows the upper section of the unit;

FIG. 6c shows the introduction of the unit to the device;

FIG. 6d shows the cooperation of the unit with the device;

FIG. 6e shows an alternative container within the device;

FIG. 6f shows a further alternative container in the device;

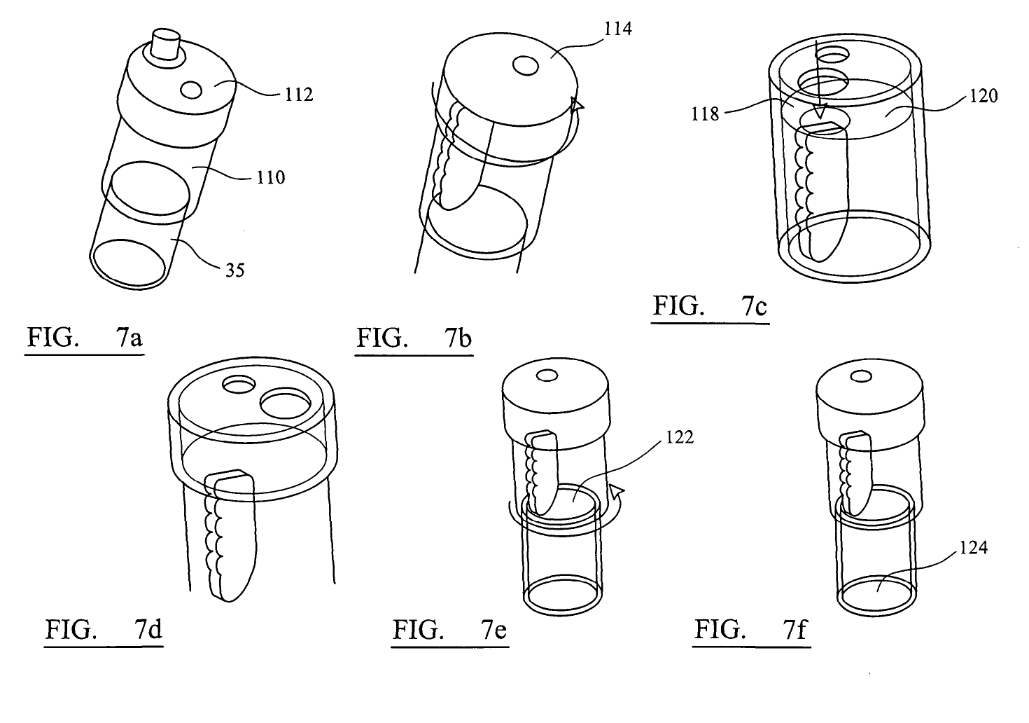

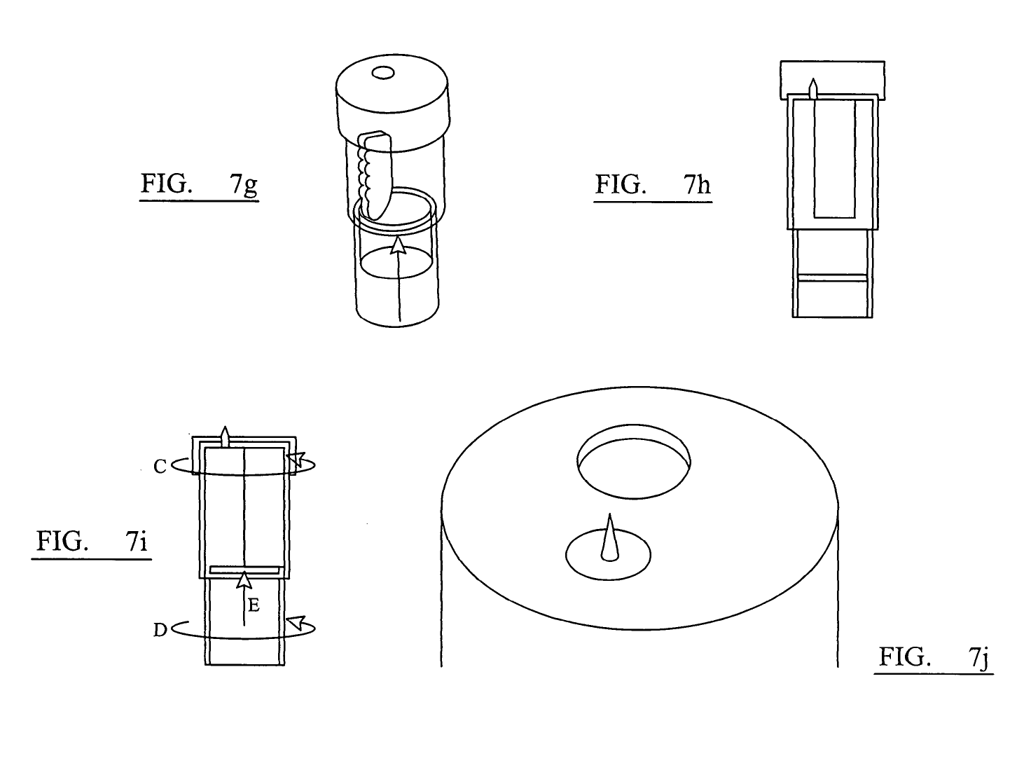

FIG. 7a shows an alternative embodiment of the container;

FIG. 7b shows the sealing of the upper section of the container of FIG. 7a;

FIG. 7c shows the upper section of the container holding the detached swab;

FIG. 7d shows the upper section of the container in the compressed state;

FIG. 7e shows a further operation stage for the container;

FIG. 7f shows a still further stage in the operation of the container;

FIG. 7g shows the transition of the container from the expanded to compressed state;

FIG. 7h shows the interaction for the restraint in this embodiment of the container;

FIG. 7i shows the transition of the container to release the needle;

FIG. 7j shows the needle piercing the diaphragm or seal;

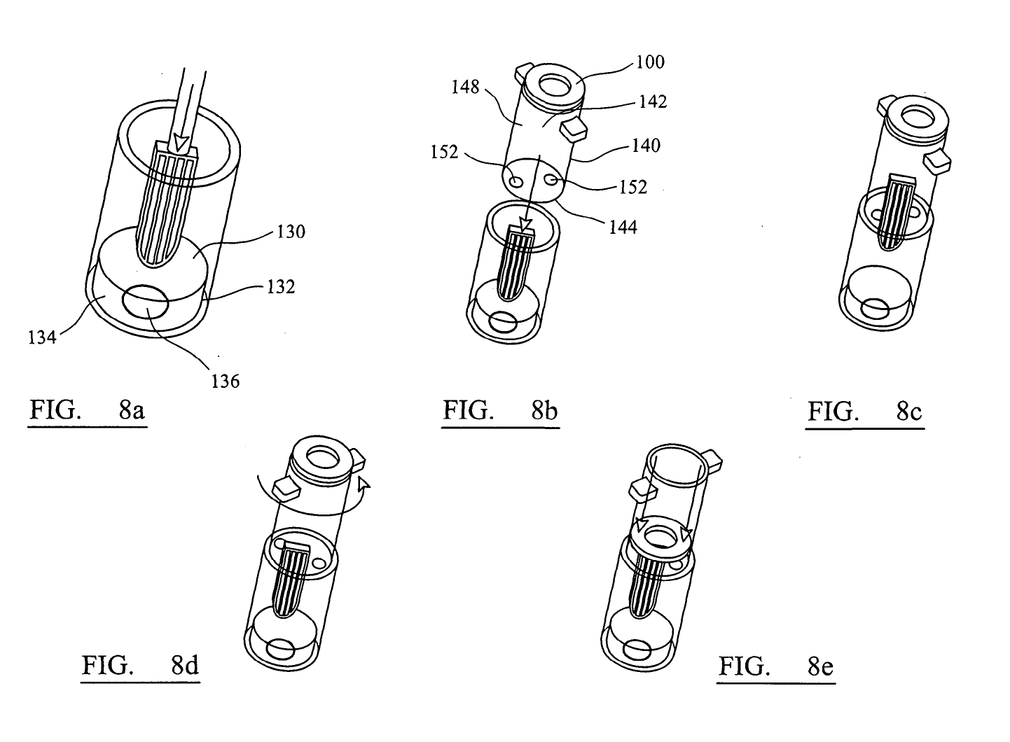

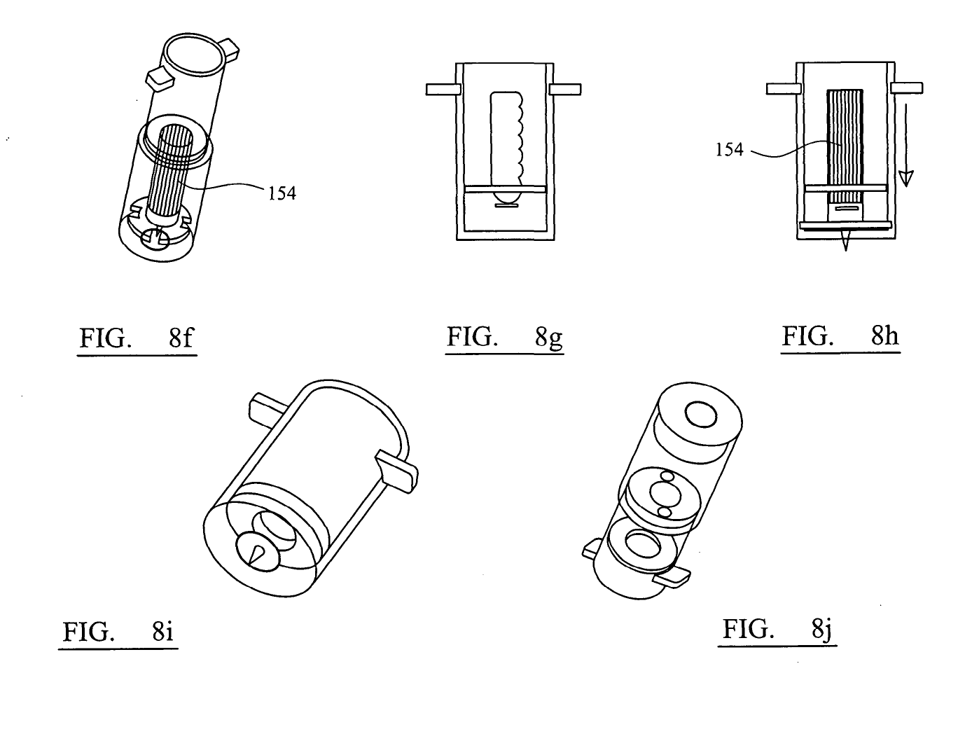

FIG. 8a illustrates an alternative embodiment of the container;

FIG. 8b shows the two parts of the container of FIG. 8a;

FIG. 8c shows the interaction of the two sections of the container;

FIG. 8d shows the release of the fluid;

FIG. 8e shows the transition from expanded state to compressed state;

FIG. 8f shows the operation of the needle;

FIG. 8g shows the transfer of the fluid into the device;

FIG. 8h shows the transition to compressed state;

FIG. 8i shows the needle penetrating the seal;

FIG. 8j is a perspective view of the base of the container;

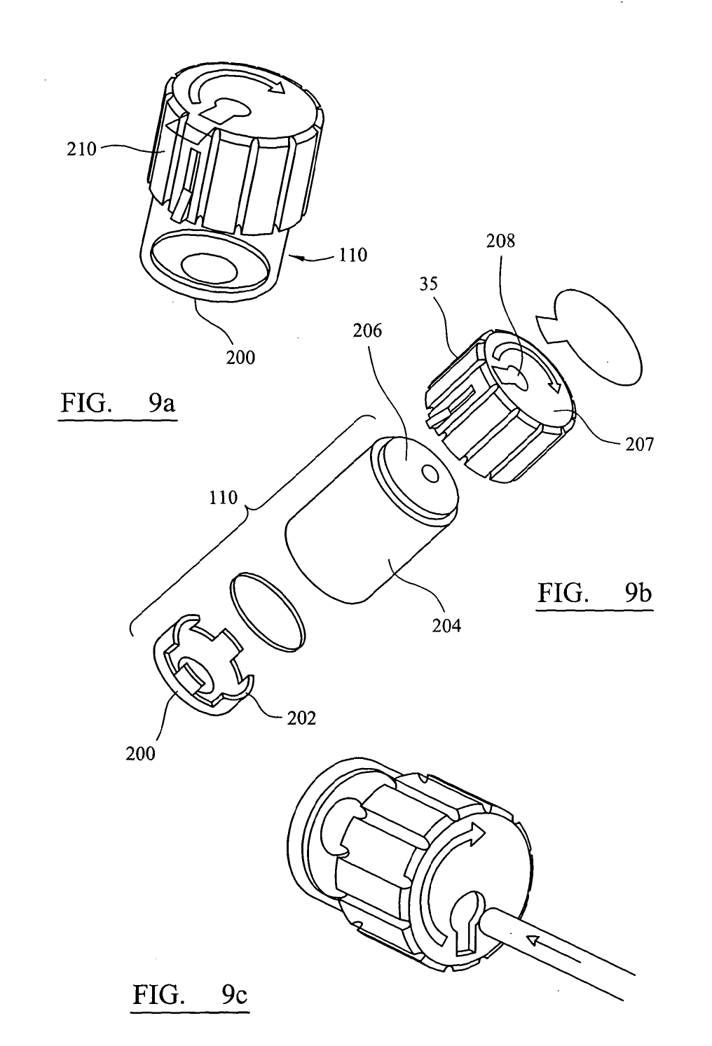

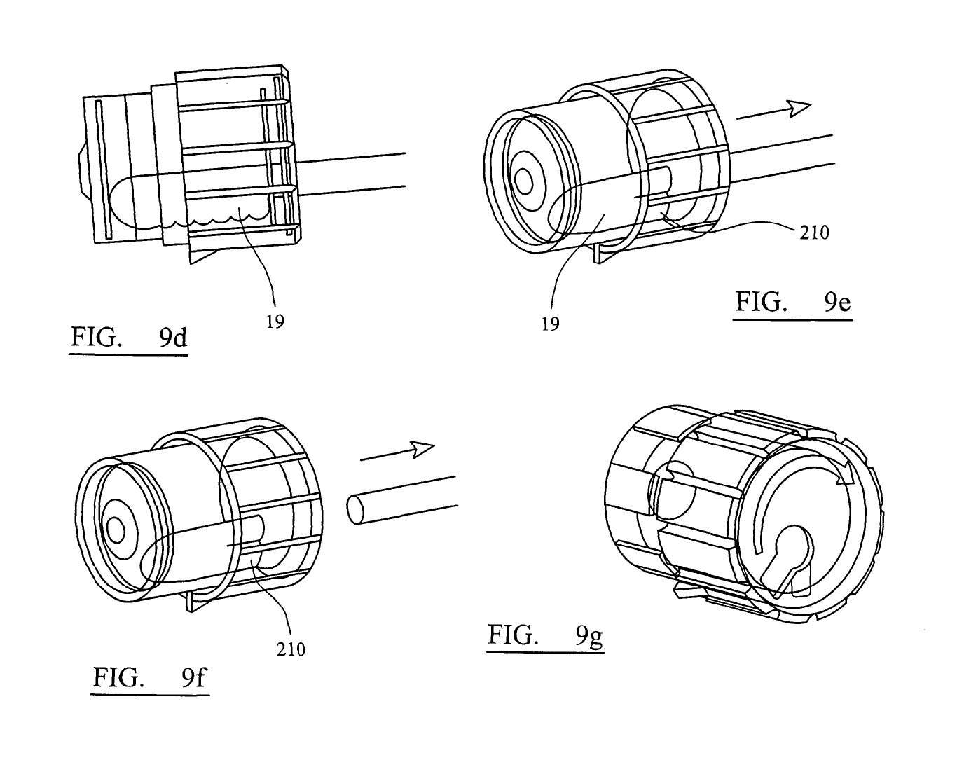

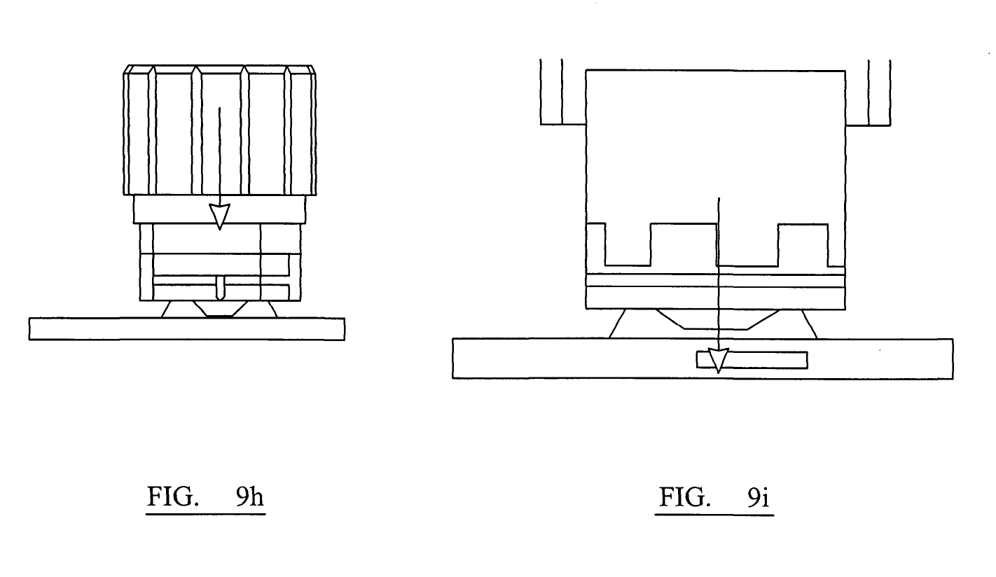

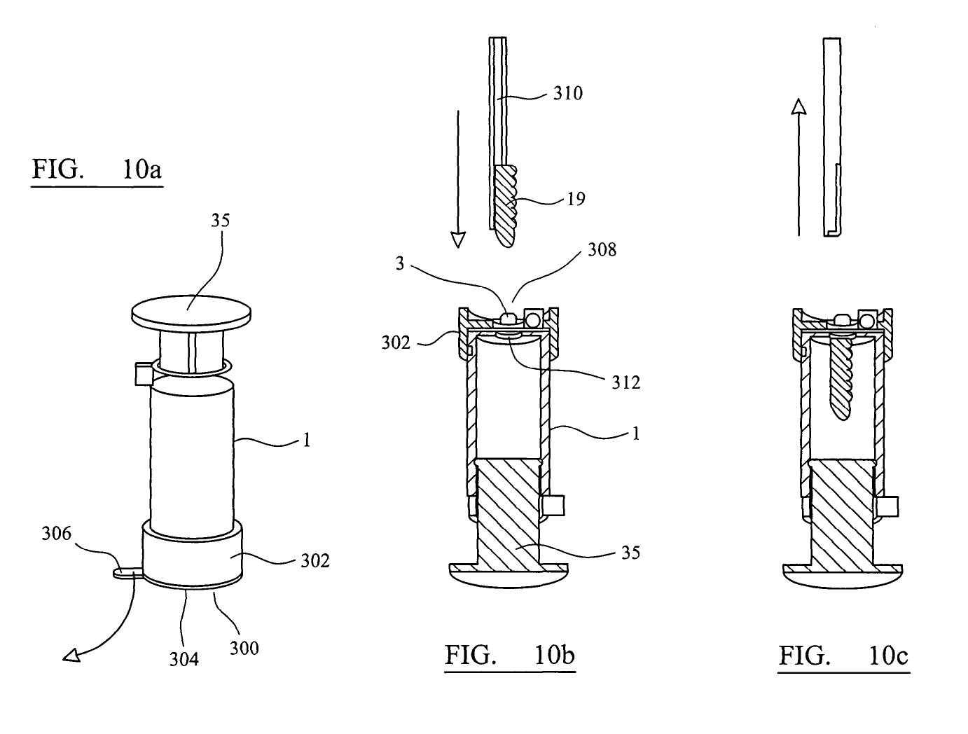

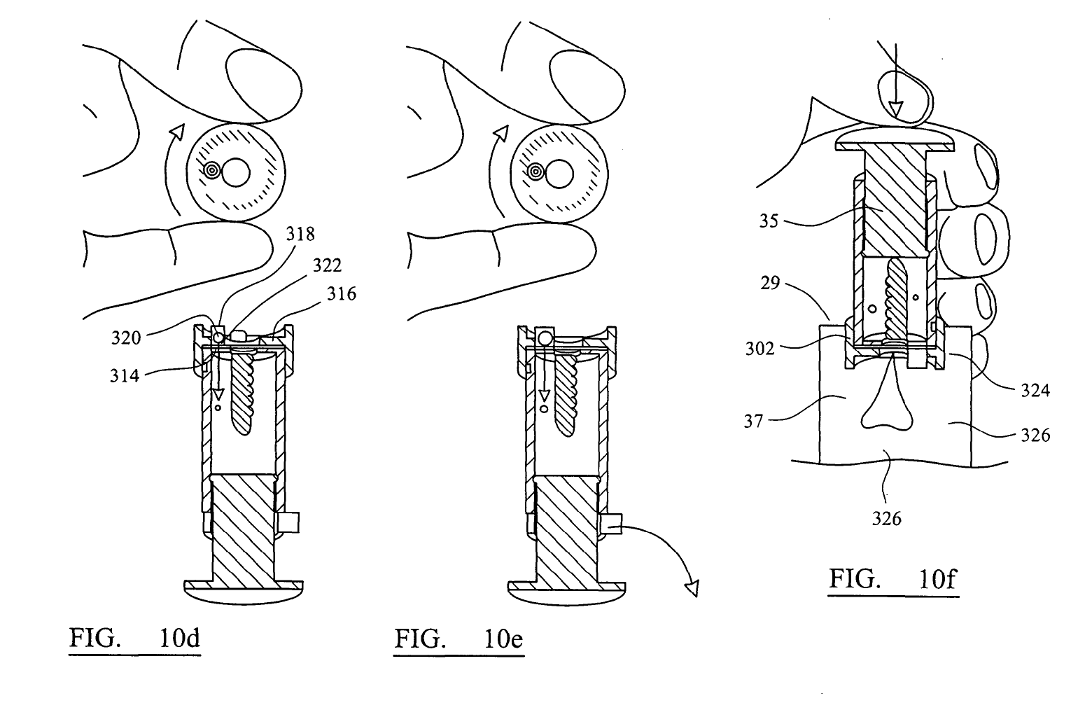

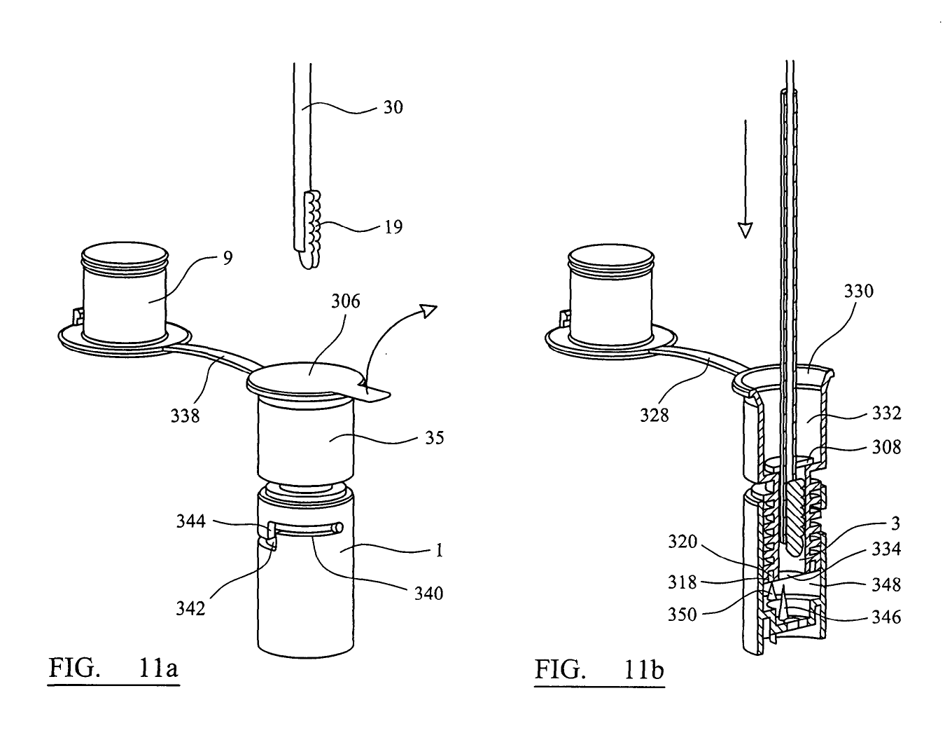

FIG. 9a is an alternative embodiment of the container;

FIG. 9b shows an exploded view of the components of the container at FIG. 9a;

FIG. 9c shows the introduction of the swab into the container;

FIG. 9d shows the swab entering the container in a side view;

FIG. 9e shows the interaction of the swab with the container;

FIG. 9f shows the retraction of the swab applicator from the container;

FIG. 9g shows the offsetting of the lid and container body;

FIG. 9h shows the interaction of the container with the device;

FIG. 9i is a detailed view of the flow path;

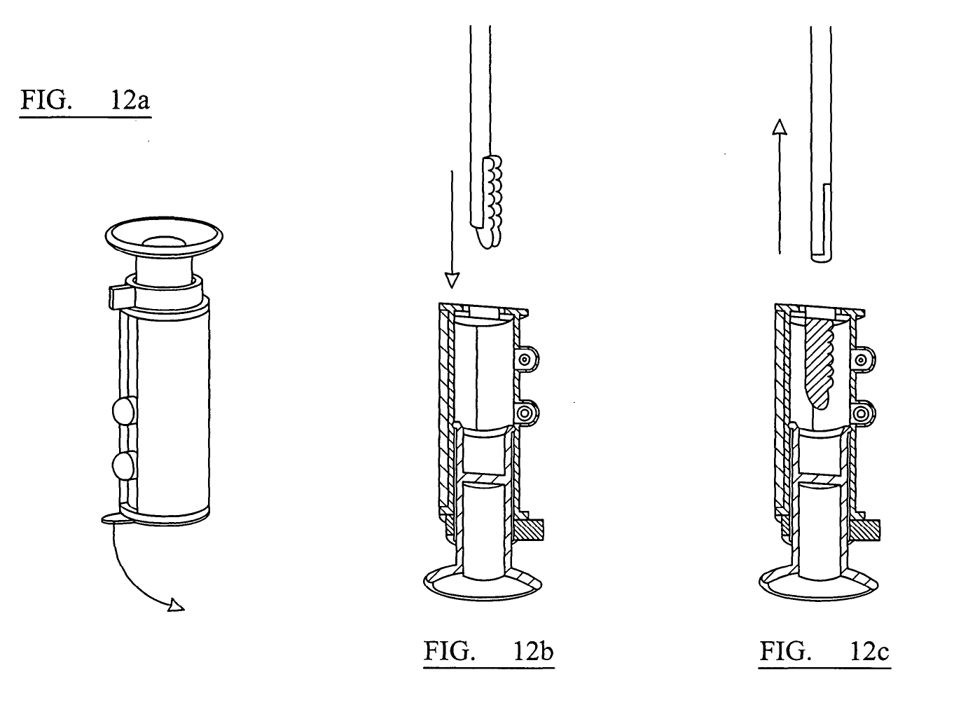

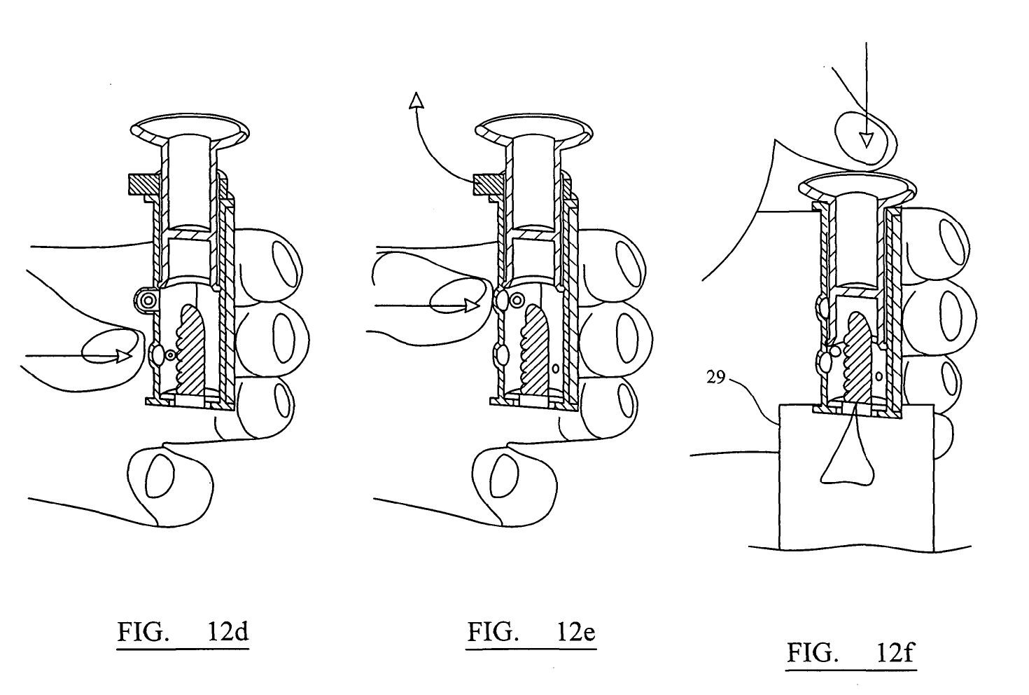

FIG. 10a is an alternative embodiment of the container;

FIG. 10b is a cross-sectional view of FIG. 10a with the swab being inserted;

FIG. 10c is a cross-sectional view of FIG. 10a after the insertion of the swab and removal of the applicator;

FIG. 10d is a cross-sectional side view and plan view of FIG. 10a showing the lysis reagent release;

FIG. 10e is a cross-sectional side view and plan view of FIG. 10a showing the bead release;

FIG. 10f is a cross-sectional side view and plan view of FIG. 10a showing the lysis fluid being dispensed from the container;

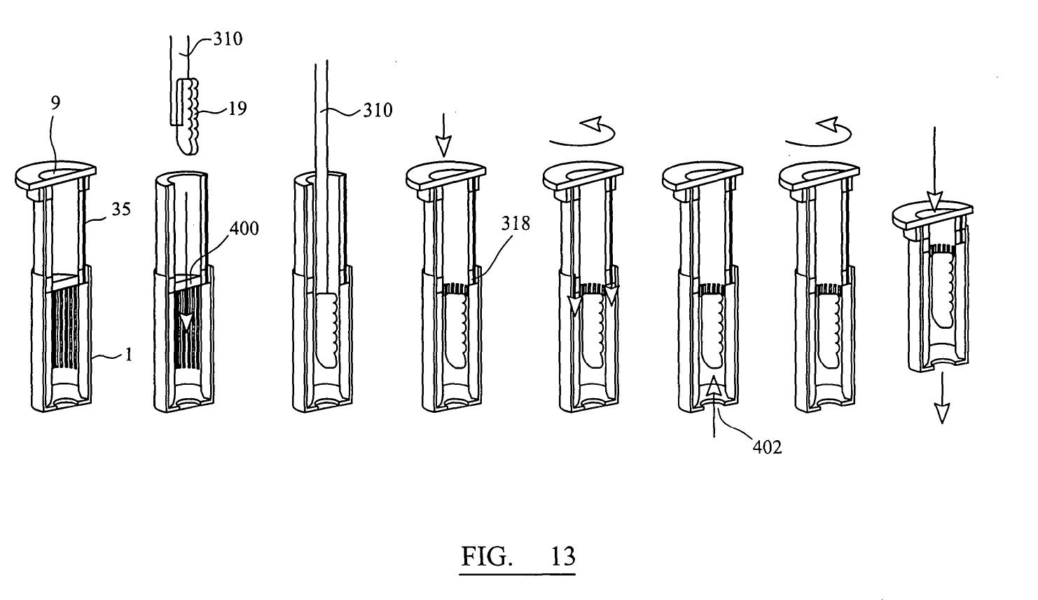

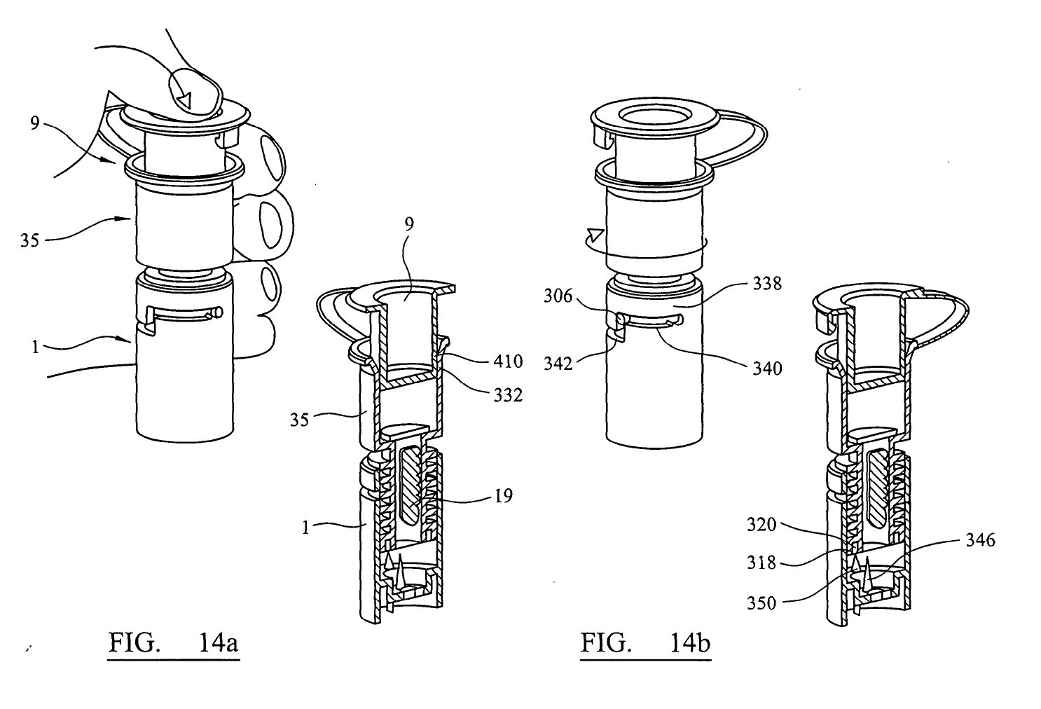

FIG. 11a is a perspective view of another embodiment being loaded;

FIG. 11b is a perspective view in cross-section of FIG. 11a showing the swab retained;

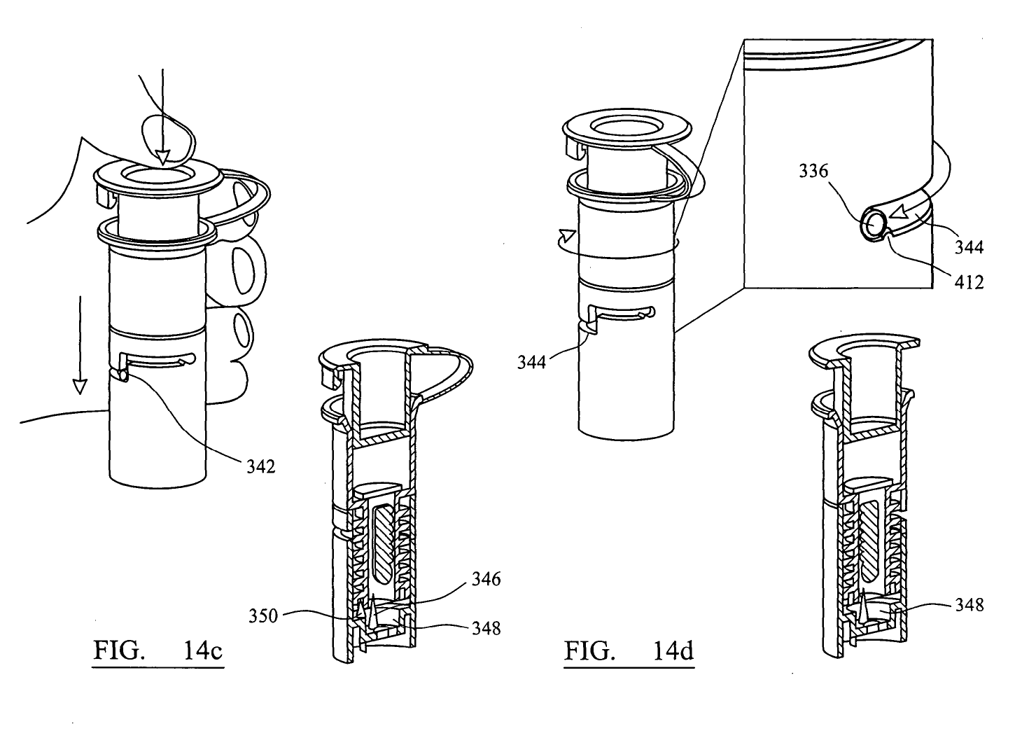

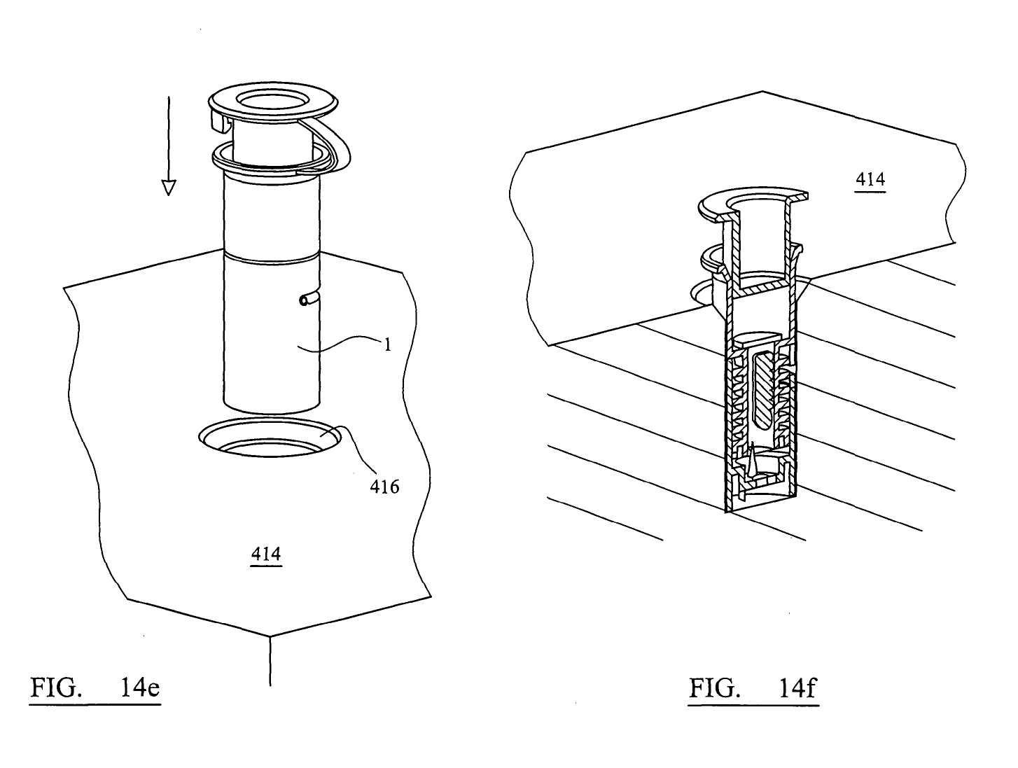

FIG. 12a is a perspective view of another embodiment with a different reagent storage and release option;

FIG. 12b is a side view in cross-section of FIG. 12a and showing the swab insertion;

FIG. 12c is the view of FIG. 12b after the swab is retained;

FIG. 12d shows the reagent release step;

FIG. 12e shows the bead release step;

FIG. 12f shows the dispense of the lysis fluid from the container;

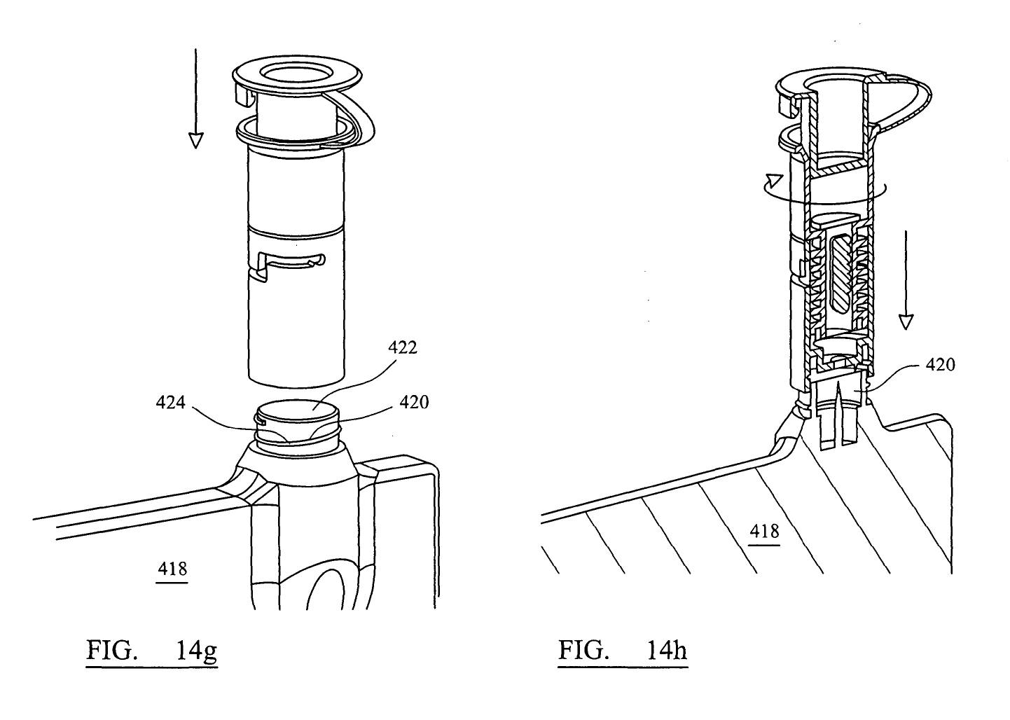

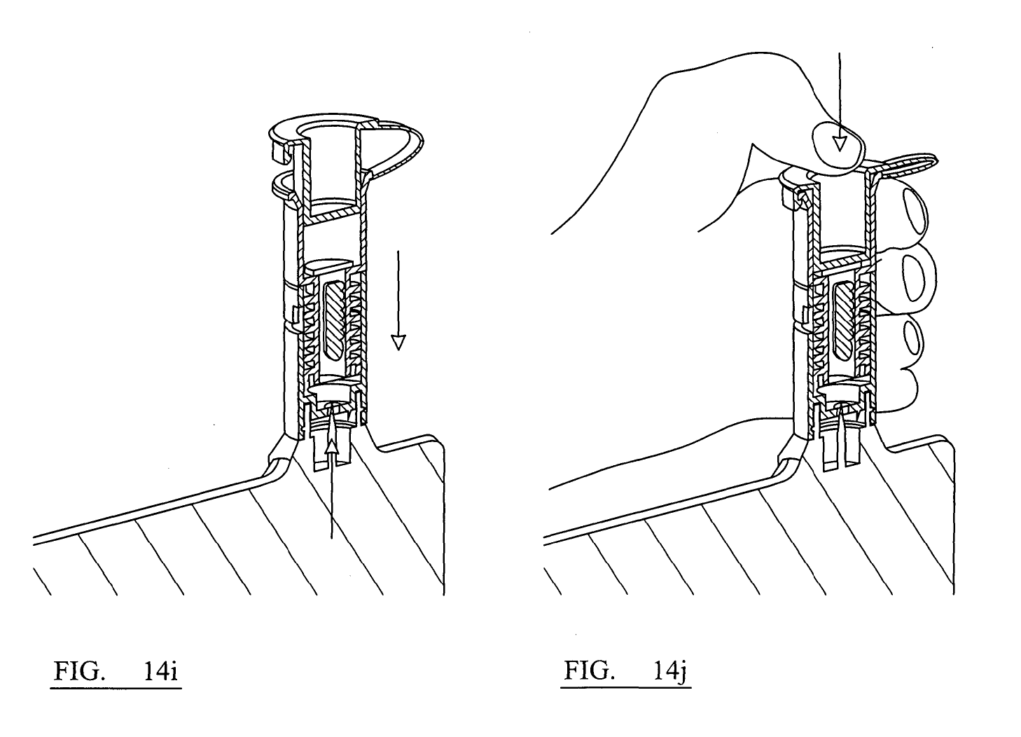

FIG. 13 shows the eight stages that an embodiment of the container goes through between the start and finish of a process;

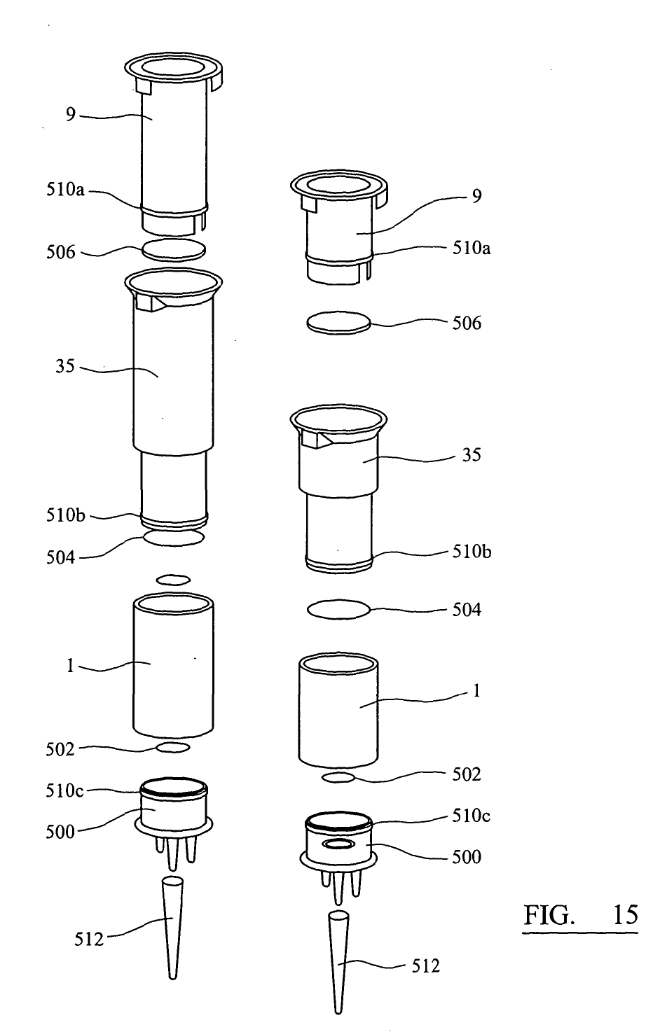

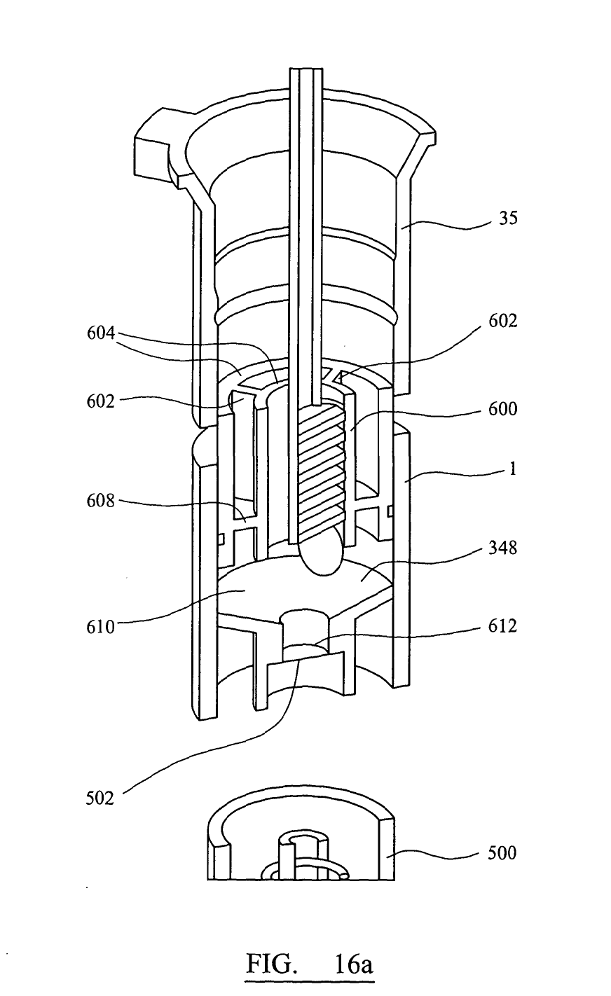

FIG. 14a shows a perspective view and a perspective cross-sectional view of a further embodiment;

FIG. 14b shows the views of FIG. 14a after rotation;

FIG. 14c shows the views of FIG. 14b after axial movement;

FIG. 14d shows the views of FIG. 14c and a detail view, after further rotation;

FIG. 14e shows the container of FIG. 14d being inserted into a heater block;

FIG. 14f shows the container, in perspective cross-sectional view in the heater block;

FIG. 14g shows the container being presented to a cartridge;

FIG. 14h shows the container being connected to a cartridge and the contents being dispensed;

FIG. 14i shows the container connected to the cartridge;

FIG. 14j shows the container's contents being dispensed;

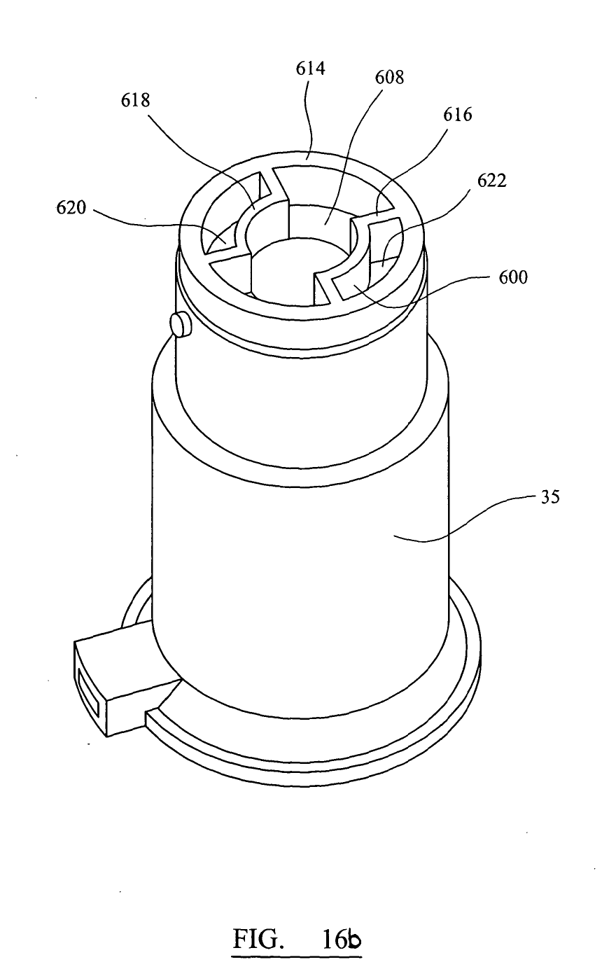

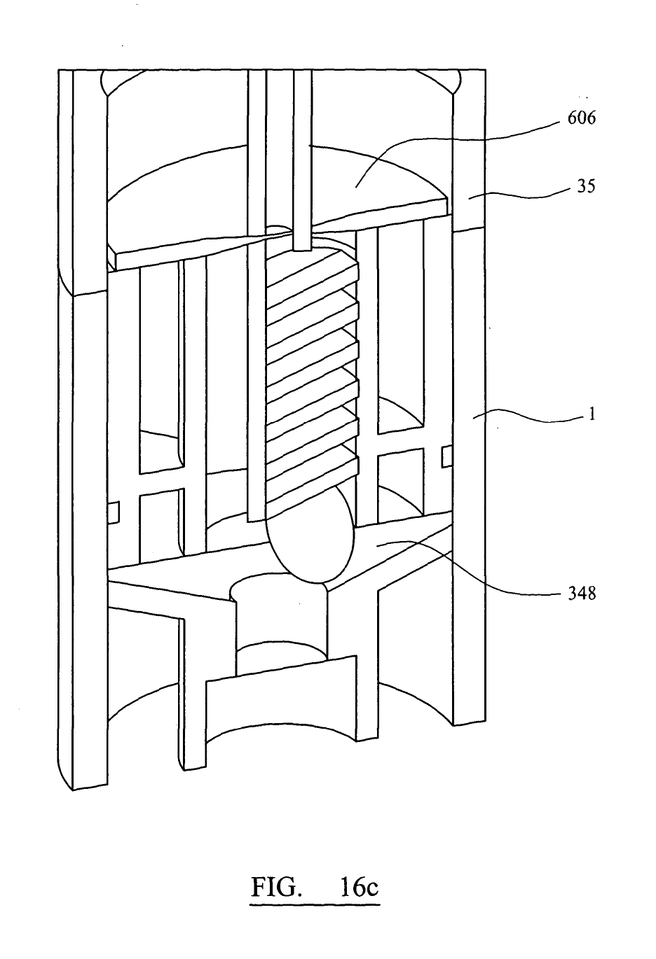

FIG. 15 is an exploded perspective view showing the components of two different size containers;

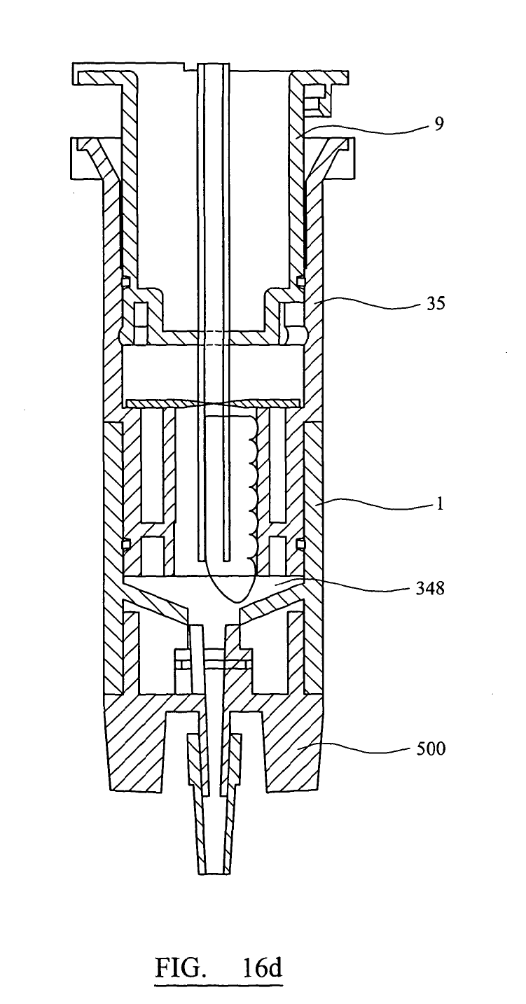

FIG. 16a is a cross-sectional perspective detail of an embodiment of the container;

FIG. 16b is a perspective view of the underside of the lid structure of the container;

FIG. 16c is a cross-sectional perspective detail of the FIG. 16a embodiment showing the swab; and

FIG. 16d shows the different components of the container of the FIG. 16a embodiment.

When analysis of a sample is required, there is a need to collect the sample from its source and/or an intermediate stage and to provide it to the analysis location or locations. This process may include one or more pre-analysis processes. The pre-analysis processes may be provided for various purposes and particularly to enable or to optimise the analysis.

An example of such a sample, is a DNA sample collected for use in forensic science. In the situation where the DNA sample is collected from an individual, it is common practice to use a swab to collect a sample from the inside of the cheek of the individual. The swab is extended to collect the sample and the retracted into a covered location.

After collection, the sample may be stored and/or transported to the processing location. Once at the processing location, generally a laboratory, a known approach involves the swab being manually taken from its storage container and manually inserted into a tube. The swab is extended in the tube to expose the swab and hence the DNA sample to liquid in the tube.

The tube and its contents are then incubated at 56° C. for 7.5 minutes. After incubation, the tube is agitated in a vortex agitator and then returned to the incubator for a further 7.5 minutes at 56° C. The resulting lysate is then extracted using a syringe and is introduced into the analysis location(s) within the analysis instrument.

The above approach faces potential issues with the exposure of the sample to contamination. The above approach requires a high level of experience and training to function correctly. The above approach is not suited to automation.

At present, forensic samples and many other biological samples, are sent from a collection location to a laboratory for processing. The results are then returned. This provides issues with the speed of response, the cost of the analysis and the ease with which analysis is available. To counteract these issues, there is a move to provide more portable and/or more widely positioned instruments capable of performing the analysis outside of a laboratory style environment. Such instruments have the potential to increase the above mentioned issues of contamination risk. It is also likely that the level of training and/or experience possessed by the operator will be lower than with laboratory style instruments and processes.

Amongst the potential aims of the present invention, therefore, is to provide a device and/or method for which the contamination risk is reduced.

Amongst the potential aims of the present invention, therefore, is to provide a device and/or method which can be successfully operated with reduced levels of training and/or experience.

Amongst the potential aims of the present invention, therefore, is to provide a device and/or method which can be successfully operated outside of a laboratory style environment.

Amongst the potential aims of the present invention, therefore, is to provide a device and/or method which is suited to automation.

The embodiments described below and the invention may provided for one or more of these or other potential aims of the invention.

The invention is illustrated by a series of embodiments which include various beneficial features and interactions. Other embodiments according to the invention can be obtained through other combinations of these beneficial features and interactions and the invention is not limited to any of the specific embodiments or uses now described.

### EMBODIMENT A

FIG. 1a shows a perspective side view of a lysis container 1. The lysis fluid 3 is provided in the lysis container 1 in advance; a pre-use state. The lysis container 1 is provided with an opening 5 in the top. The opening 5 is sealed by a foil seal 7. The inside of the lysis container 1, and hence the lysis fluid 3, are protected against contamination in this form.

The opening 5 has a first state, as shown, in which the lid 9 is spaced from the opening 5. A spur 11 connects the lid 9 to the lysis container 1.

On the lid 9 are two foil seals 13a and 13b. One of the foil seals 13a is positioned at the mouth of the lid 9. The other foil seal 13b is positioned on the outside of the end of the lid 9 and covers a rubber cap 14.

The foil seal 7 on top of the lysis container 1 is peeled away to remove it; the use state. The rubber diaphragm or seal 15 provided under the foil seal 7 is thus revealed. The rubber diaphragm or seal 15 has a slit 17 provided in it. As shown, the slit 17 is in a first closed state; the slit 17 acting as a valve to retain the lysis fluid 3 in the lysis container 1 in the first closed state.

As shown in FIG. 1c, the lysis container 1 is held upright and insertion of the swab 19, on the end of the swab applicator 21, commences. The end 23 of the swab 19 is positioned on the rubber diaphragm or seal 15 in contact with the slit 17. The swab applicator 21 is pushed towards the lysis container 1 such that the swab 19 and/or swab applicator 21 cause the slit 17 to move to a second open state and thereby allow the passage of the swab 19 and swab applicator 21 into the lysis container 1, as shown in FIG. 1d.

Withdrawal of the swab applicator 21 is then provided. The rubber diaphragm or seal 15 ensures that the slit 17 closes as far as possible around the swab applicator 21 during insertion and removal. This minimises or prevents lysis fluid 3 leaking. When the swab 19 contacts the inside of the rubber diaphragm or seal 15, the resistance provided helps to pull the swab 19 out of its engagement with the end of the swab applicator 21. The result is that the swab applicator 21 is fully withdrawn from the lysis container 1 and the swab 19 is retained within the lysis container 1. With the swab applicator 19 withdrawn, the slit 17 assumes the first closed position once more, see FIG. 1e.

The lid 9 is now separated from the lysis container 1 by breaking the spur 11. The foil seal 13a is then removed. The lid 9 can then be placed over the opening 5 to provide the opening in the second position; sealed use state. The rubber cap 14 provided within the lid 9 provides additional sealing for the opening 5, against lysis fluid 3 leakage, during subsequent operations. The rubber diaphragm or seal 14 is free from any slits or other apertures. The foil seal 13b is still in position on the outside surface of the rubber cap 14 and lid 9, see FIG. 1f.

With the sample safely in the lysis container 1, the sealed use state can be taken on to the initial process state. In the initial process state, the lysis container 1 is taken and introduced into a heating and agitation device. The heating and agitation device can be a separate device from the analysis device or can be a part of the analysis device itself. The heating and agitation device provides conditions which, in conjunction with the lysis fluid 3, promote release of the DNA sample from the swab 19 into the lysis fluid 3.

Once completed, the initial process state can be taken onto the loading state. In basic terms, the analysis is performed by a device and it is necessary to introduce the lysis fluid and hence sample into that device. The device itself can take many possible forms and these forms are not restricting on the present invention.

At the start of the loading stage, FIG. 2a, the container is in an initial loading state. The foil seal 13b is removed from the lid 9 so as to expose the rubber diaphragm or seal 14. A seal 25 on the inlet 27 to the device 29 is also removed. The lysis container 1 can then be pushed into the inlet 27. The external perimeter of the lysis container 1 and the external perimeter of the inlet 27 are profiled to snugly accommodate one another, FIG. 2b.

The insertion passes the container through the physical arrangement shown in FIG. 2c. The rubber cap 14 as it advances into the inlet 27 contacts an inlet rubber cap 31 provided within the inlet 27. Behind this inlet rubber cap 31 is a needle 33. Further insertion beyond the position shown in FIG. 2c results in the rubber cap and inlet rubber cap being pushed against and pierced by the needle 33. As a result, a fluid path from the inside of the container 1 into the device 29 is provided.

Even when fully inserted into the inlet 27, a section 35 of the container 1 still projects beyond the perimeter 37 of the device 29. This section 35 is capable of being reduced in volume from an expanded state to a compressed state. In the pre-use state, use-state, sealed use state, initial process state and initial loading state, as shown in FIGS. 2a and 2b, the section 35 is in the expanded state. To provide for the transition from expanded to compressed state, the resilient band 39 which is partially wrapped around this section 35 is removed. The resilient band 39 resists any attempt to move from the expanded state to the compressed state with the band 39 in place. As a result premature dispensing of the fluid and the sample from the container 1 is avoided.

The section 35 is provided with a concertina type profile to allow for the transition from expanded to compressed state. By applying force to the end surface 41 of the container 1 towards the device 29, the section 35 is reduced in volume and the lysis liquid is pumped into the device 29. The section 35 is restrained in the compressed state to prevent its reversing to the expanded state. This can be achieved by the interaction of one or more components on the container 1 with one another and/or interaction between one or more components on the container 1 with one or more components on the device 29. Maintaining the section 35 in the compressed state avoids any undesirable vacuum being generated which could withdraw fluid from the device 29. Retaining the container 1 in the inlet 27 means that a seal is provided against contamination and the like.

### EMBODIMENT B

A second embodiment of the invention is illustrated with the assistance of FIGS. 3a to f.

In this embodiment, the container 1 has many of the features of the first embodiment and these features are not described again here. The operation is the same as described above for FIGS. 1a through 1f. The variations relate to the protection of the section 35 against inadvertent compression and the manner in which the container 1 interacts with the device 29.

As illustrated in FIG. 3a, the section 35 again has a concertina profile 50. The movement of the end surface 41 towards the remained of the container 1 and hence the transition of the section 35 from expanded to compressed form is resisted differently.

Attached to the ends 52 of the end surface 41 are bridging elements 54. The end 56 of the rigid part of the container 1 is provided with a pair of protrusions 58. These, as shown in FIG. 3e, cooperate with the ends 60 of the bridging elements 54. In this cooperating position, the bridging elements 54 resist movement of the end surface 41 towards the rest of the container 1.

During insertion of the container 1 into the inlet 27 in the device 29, protrusions 62 within the inlet 27 deflect the ends 60 of the bridging elements 54 outwards and out of cooperation with the protrusions 58. As a result, the bridging elements 54 no longer restrain movement of the end surface 41 relative to the rest of the container 1. The section 35 can, therefore, be compressed so as to drive the fluid from the container 1 into the device 29.

The second embodiment also illustrates a more automated approach to the initial processing and loading states.

In FIG. 3b, the container 1 is shown inserted into a slot 64 associated with a heating and agitation state and provided as a part of the device 29. As shown, heat and agitation are applied in this position, as necessary. The container 1 is then brought into the necessary position relative to the analysis components 66 of the device 29 by a series of servo motors, not shown. Once the necessary alignment is reached via rotation, as shown in FIG. 3c, the container 1 is forced towards the analysis components 66. The interaction to provide the fluid path is as described previously.

### EMBODIMENT C

A third embodiment of the invention is illustrated with the assistance of FIGS. 4a to c.

The container 1 has many of the same features as described previously and the description of those features is not repeated here. The principle differences relate to the form of the section 35 which has the expanded and compressed states and provides for the transfer of the fluid into the inlet 27 and/or device 29.

In this embodiment, the section 35 is in the form of a chamber 70 with a dome 72 projecting away from the rest of the container 1. The chamber 70 is effectively a blister. The container 1 is inserted into the inlet 27 in this form, FIGS. 4a and 4b. Force is then applied to the dome 72 and this causes the dome 72 to collapse and thereby reduce the volume of the section 35. In the preferred form shown, the dome 72 effectively becomes over centred so as to restrain it in the position whereby the section 35 is in the compressed state.

### EMBODIMENT D

The fourth embodiment is more materially different from the previous embodiments than they are from each other and is illustrated with reference to FIG. 5a to e.

The container 1 is in the form of a right cylinder with a lid 9 which is removed by unscrewing in a conventional manner. Within the container 1, the lysis fluid 3 is already present. With the lid 9 removed, it is possible to insert a swab 19, FIG. 5b. The swab 19 is inserted such that the it passes into the midst of a ring 80 of protrusions 82 which extend from the base or wall of the container 1. Gaps exist between the protrusions 82 such that the lysis liquid 3 has full opportunity to contact the swab 19. The relative dimensions of the ring 80 and swab 19 are such that at least some of the protrusions 82 are deformed by the insertion of the swab 19. The resilience of the protrusions 82 means that this results in a clamping force being applied to the swab 19 by the deformed protrusions 82. When the swab applicator, not shown, is removed this results in the swab 19 being detached and retaining in the container 1. This is shown in FIG. 5c, with only the top section of the container 1 actually shown.

With the swab 19 inserted and retained, the lid 9 is returned and the container is sealed fully.

As shown in FIG. 5d, the base 86 of the container 1 features a rubber diaphragm or seal 88 which at this state is covered by a removable foil seal 90 so as to protect the rubber diaphragm or seal 88 against contamination.

Once again, from the sealed use state the container 1 is taken through a initial processing state, commonly heating and agitation, and then to a loading state, FIG. 5e. In the loading state, the removable foil seal 90 is removed. The seal 92 provided over the inlet 27 is also removed.

The container 1 is then pushed into the inlet 27, FIGS. 5f and 5g, until the base 86 abuts the bottom 98 of the inlet 27.

As the container lis pushed into the inlet 27, a needle 90 in the bottom of the inlet 27 pierces the diaphragm or seal 88 to provide the fluid path, FIG. 5g. As with the other embodiments, the container 1 includes a section 35 which has an expanded state, as shown in the Figures up to this point, and a compressed state. To enable the section 35 to be moved to the compressed state, the upper section 35 of the container 1 is rotated clockwise, FIG. 5h. During the rotation, the lower part of the container 1 is held in position by the device 29. The rotation brings alignment between one or more shafts on one part of the container 1 and one or more projections on the other part of the container 1. The parts may be the upper and lower sections or vice versa. Each projection is able to slide in the shaft and so the end surface 41 of the container 1 is brought closer to the base 98 of the inlet 27, FIG. 5i. The section 35 of the container 1 slides inside the other section, FIG. 5j and k. The ring 80 of projections 82 holds the swab 19 so that it does not obscure the flow path. The end result is the transition of the section 35 to the compressed state and hence the fluid 3 is driven into the device 29. The section 35 is held in this position to prevent the section 35 expanding and reversing the process.

### EMBODIMENT E

In this embodiment, a container 1 of the type described in Embodiment D is used. The container 1 is loaded with the swab as described above.

Once in the sealed use state, the container 1 is placed inside a process unit 100. The inside of the unit 100 is accessed via opening door 102 provided on a hinge 104. The inside of the unit 100 is configured to correspond in profile to the container 1 so as to hold it in the correct position, FIG. 6a. The unit 100 provides for the heating and agitation of the container 1.

The unit 100 is a readily portable, hand held unit. The shape and configuration is similar to a pen.

The fluid within the container 1 is dispensed into the device 29 direct from the unit 100.

The unit 100 has a rotatable top part 106, FIG. 6b. The top part 106 is also capable of being depressed. Clockwise rotation of this part 106, followed by depression is used to start the agitation and heating, the initial processing state.

Once the liquid 3 is ready for injection into the device 29, the aperture 104 in the end of the unit 100 is aligned with a protrusion 106 on the device 29, FIG. 6d. The protrusion 106 has a rubber diaphragm or seal seal. Further rotation of the top part 102 of the unit 100 allows the section 35 of the container 1 to undergo the transition from the expanded state to the compressed state. This compression includes causing a needle, not shown, in the container 1 to pierce the seal to complete the flow path into the device 29. Various arrangements to provide this approach are described in more detail in the embodiments that follow.

FIG. 6e and f illustrate the use of the pen style unit 100 with different container 1 embodiments.

### EMBODIMENT F

In this embodiment, a container 1 of a different form is used. The container has an upper cylindrical section 110 of greater diameter than the lower cylindrical section 35. Provided on the upper section 110 is a lid 112. The lid 112 can be rotated on the upper section 110 to provide various functions described in more detailed below. The lid 112 has an opening 114 covered by a removable foil seal 116. With the seal 116 removed, it is possible to access the inside of the container 1 through the opening 114 and an aligned opening in the upper section 110. The swab 19 can thus be put in the container 1.

As shown in FIG. 7b, with the swab 19 in position in the upper section 110 of the container 1, the lid 112 is rotated anti-clockwise one stop so as to offset the opening 114 through the lid 112 relative to the opening through the upper section 110. This results in the sealing of the container 1.

FIG. 7c shows the internal structure of the upper section 110 of the container 1 in more detail and the functionality offered prior to the rotation of FIG. 7b. Within the upper section 110 a structure 118 is provided. The structure 118 includes a series of radially extending protrusions 120. When inserted, the swab 19 causes the protrusions 120 to deform. The resilience of the protrusions 120 urges them back to their resting state and therefore applies a restraining force to the swab 19 when the swab applicator 21 is removed. This results in the detachment of the swab 19 from the swab applicator 21 and the retention of the swab 19 within the container 1. The protrusions 120 maintain the swab 19 in the upright position. The structure 118 is also provided with an injection needle 33. The injection needle 33 is positioned below the level of the lid 112, but has the potential to be moved up and through the lid 112, as described later.

Referring to FIGS. 7d and 7e, relative rotation of the upper section 110 and lower section 35 causes the rupture of a seal 122 provided between the upper section 110 and the lower section 35. This allows contact between the lysis fluid 3 held in the lower section 35 with the swab 19 in the upper section 110. The movement of the plunger 124 up the inside of the lower section 35 towards the upper section 110 is now possible. This forces the lysis fluid 3 into the upper section 110. As a result, the container moves from the expanded state of FIGS. 7a, 7b and 7c to the compressed state of FIG. 7d etc.

FIG. 7d shows more clearly the interaction between the lid 112 and the upper section 110 of the container 1. The lid 112 is in effect an open cylinder with a diameter greater than the upper section 110 of the container 1. Different relative alignments between the lid 112 and the upper section 110 allow different operations.

FIG. 7h illustrates an abutment provided within the container 1 prevents the lower section 35 being advanced into the upper section 110. This restraint and the positioning of a solid part of the lid 112 over the needle 33 prevents the needle 33 being deployed. The solid part of the lid 112 provides a non-pierceable part of the lid 112 aligned with the axis of the needle.

With the device in this state, the initial processing state, for instance heating and agitation of the container, can be provided.

Subsequent to this, and as illustrated in FIG. 7i, the lid 112 is first rotated through a further stop relative to the upper section 110. As a result, a rubber diaphragm or seal 126 provided over an opening in the lid 112 is brought into alignment with the axis of the needle 33. A further rotation of the lower section 35 relative to the upper section 110 removes the alignment between the restraint between the upper section 110 and lower section 35. This allows the lower section 35 to be advanced into the upper section 110; the transition from expanded to compressed state. This causes the displacement of the needle 33 through the rubber diaphragm or seal 126. This provides the fluid pathway into the device, not shown. The compression also applies pressure to the fluid within the upper section 110 of the container 1 and hence advances the fluid along the flow path and into the device. FIG. 7j shows a detailed perspective view of the needle 33 having pierced the diaphragm or seal 126.

### EMBODIMENT G

In this embodiment, container 1 of an alternative structure described previously is provided, but with similarities to Embodiment F described above.

FIG. 8a shows the insertion of the swab 19 into the first section 110 of the container 1. During insertion, the swab 19 engages with a ring of projections of the type previously mentioned. These projections provide a clamping force to the swab 19 and so retain it within the first section 110 when the swab applicator 21 is withdrawn. The projections are mounted on a structure 130 supported by the wall 132 of the section 110. The base 134 of the section 110 includes a rubber diaphragm or seal 136 with a removable seal provided over the outside surface.

Once the swab 19 is in position, the second section 35 of the container 1 is introduced, FIG. 8b. This is a further right cylinder 140 which is sized to be received within the profile of the section 110 carrying the swab 19. Within the right cylinder 140 is a further right cylinder 142. The two cylinders 140, 142 are coaxial and are provided with a linking base 144. The inner cylinder 142 is provided with a through passage 146 sized to allow the passage of the received swab 19 and the projections that retain the swab 19, when the section 35 is pushed down into the section 110.

The annular space 148 defined between the outer cylinder 140, base 144 and the inner cylinder 142 is sealed by an annular plunger 150. Two further seals 152, in the form of rubber diaphragm or seals, are provided in the base 144. The lysis fluid 3 is stored in the annular space 148.

The section 35 is pushed into the section 110 is retained therein, for instance by a snap fit engagement. Rotation of the section 35, FIG. 8d, causes the seals 152 to break and release the lysis fluid 3 into the section 110 which contains the swab 19.

The annular plunger 150 is then forced down the annular space 148, FIG. 8e, so as to displace the lysis fluid 3 into the section 110 containing the swab 19; the transition from expanded to contracted state. This also causes the pressure within the container 1 to be elevated, with that pressure being used at a later point to displace the fluid along a flow path and into the device, not shown.

Following this stage, heating and agitation is provided.

As shown in FIG. 8f, further rotation of the sections 35, 110 relative to one another then allows the structure 154 incorporating the swab 19, projections and the needle 33 to be depressed. This forces the needle 33 through the seal 136 in the bottom of the section 110. This releases the pressure in the container 1 and that pressure drives the lysis fluid 3 into the device.

If necessary, the section 35 can also now be depressed further within the section 110 so as to supplement the pressure for injecting the lysis fluid 3 into the device.

It is possible to provide a return mechanism, such as a spring loading, so as to withdraw the needle 33 into the container 1 once dispensing has been completed. This prevents the needle 33 being a safety hazard due to its projecting outside of the container 1.

FIG. 8i shows the needle having penetrated the diaphragm or seal 136 in the base of the container 1. FIG. 8j shows a perspective view of the overall container 1 viewed from the bottom.

### EMBODIMENT H

In this embodiment, a container 1 is provided which is intended for automated processing by the device 29.

The container 1 is shown in perspective view in FIG. 9a. The lysis liquid and other reagents can be provided within the container 1.

The container 1 is in the form of a cylindrical section 110 which is closed at one end by a rubber diaphragm or seal 200. As shown in FIG. 9b, the diaphragm or seal 200 is mounted in the centre of an end wall provided by a cap 202 which is a snap fit on to the end of the open cylinder 204 The other end is also closed by a diaphragm or seal 206.

The container 1 also includes a section 35 which is effectively a cap over the section 110. The end surface 207 of the section 35 includes a key shaped opening 208. The end surface 207 is protected by a tear off seal 110.

To use the container 1, the tear off seal 110 is removed so as to expose the opening 208. As shown in FIG. 9c, the swab 19 on its applicator 21 can be aligned with the opening 208 and pushed forward so as to penetrate the seal 206 and enter the container 1. The swab 19 within the container is shown in the side view of FIG. 9d. A slit 210 is provided on the seal 206 so as to receive the swab 19 and applicator 21, but resist the withdrawal of the swab 19 so as to retain the swab 19 in position. This part of the process is shown in FIGS. 9e and 9f. The swab 19 is naturally received within the lower part of the container 1 due to gravity and is thus submerged in the lysis fluid 3.

With the swab 19 loaded, the section 35 can be rotated through 180° relative to the section 110. The rotation causes the opening 208 to be offset relative to the seal 206, thereby supplementing the sealing provided, FIG. 9g.

Heating and agitation may now be provided to the container 1.

To load the device 29, the base 202 with the seal 200 on the section 110 is brought into contact with the opening into the device 29, FIGS. 9h and 9i. Further rotation of the section 35 relative to the section 110 allows the section 35 to be advanced axially down over the section 110. The same force which moves the container 1 from the expanded to the contracted state also pushes the container 1 against a needle and causes the seal 200 to be punctured and a flow path into the device to be formed. The pressure within the container 1 assists in driving the fluid into the device 29.

### EMBODIMENT I

The next embodiment is illustrated with reference to FIG. 10a to f.

The container 1 is in the form of a right cylinder which accommodates a section 35, in this case a plunger, in an expanded state. The other end 300 of the container 1 is provided with a base section 302. The annular face 304 of the base section 302 is provided with a removable foil cover 306.

As shown in FIG. 10b, with the cover 306 removed, the inside 308 of the base section 302 is accessible. This allows a swab 19 on a swab applicator 310 to be inserted such that it passes into the container 1 through a location 309 mounted on diaphragm or seal 312.

The relative dimensions of the slot and swab 19 are such that when the swab applicator 310 is removed this results in the swab 19 being detached and retaining in the container 1. This is shown in FIG. 10c.

With the swab 19 inserted and retained, the base section 302 is rotated relative to the container 1. This results in a through aperture 314 in the annular end wall 316 of the container 1 being aligned with a holding location 318. The holding location 318 encloses one or more beads 320 on all sides, apart from an opening 322 which faces the annular end wall 316 of the container 1. A similar approach is used to release the lysis reagent 3 by rotating the base section to give alignment of the holding location and through aperture.

Prior to rotation of the base section 302, the annular end wall 316 serves to retain the one or more beads 320 within the holding location 318. Rotation and hence alignment of the holding location 318 with the through aperture 314 causes the one or more beads 320 to fall into the container 1.

As a result of these steps, FIG. 10f, the DNA on the swab 19, the lysis fluid 3 and the one or more beads 320 are all in contact with one another. This allows for the DNA to be released from the swab 19, lysed by the lysis fluid 3 and then become fixed to the one or more beads 320, for instance by virtue of the surface chemistry of the one or more beads 320 compared with that of the lysed DNA.

Once again, from the sealed use state the container 1 is taken through a initial processing state, commonly heating and agitation, and then to a loading state, FIG. 5f. In the loading state, the base section 302 is pushed into engagement with the device 324. In this case, the base section 302 is a male part received within a female part 324 provided on a cartridge 326 or other location into which the sample is to be dispensed.

As the container lis pushed into the female part 324, an inlet 27 on the cartridge 326 is aligned with the slit in the rubber diaphragm or seal 308.

As with the other embodiments, the container 1 includes a section 35 which has an expanded state, as shown in the Figures up to FIG. 10f, and a compressed state. To enable the section 35 to be moved to the compressed state, the upper section 35 of the container 1 is rotated clockwise. During the rotation, the lower part of the container 1 is held in position by the device with which it is engaged. The rotation brings alignment between one or more openings on one part of the container 1 and expanded section 35 and one or more projections on the other part of the expanded section 35 and/or container 1. The end result is the transition of the section 35 to the compressed state and hence the fluid 3 is driven into the device 29. The section 35 is held in this position to prevent the section 35 expanding and reversing the process.

### EMBODIMENT J

A further embodiment is shown in FIG. 11a and 11b. In this case, the upper section 35 again has an expanded state, as shown in FIG. 11a, and the ability to move within the container 1.

A lid 9 is provided completely outside of the upper section 35, but connected there to by a flexible attachment 328.

The opening 330 into the upper section 35 is sealed by a removable foil cover 306.

With the cover 306 removed, it is possible to insert the swab 19 on the swab applicator 310 into the upper section 35. This includes passing the swab 19 through the mouth section 332 of the upper section 35 and through a slit in a diaphragm or seal 308 and into the lysis fluid 3 within the upper section 35. The base of the upper section is sealed by a membrane 334.

The swab 19 is retained in this position when the swab applicator 210 is removed. Hence, lysis of the DNA begins and the DNA is removed from the swab 19.

Rotation of the upper section 35 relative to the container 1, causes pin 336 to move along track 338. The track 338 has a circumferential section 340, an axial section 342 and a further circumferential section 344. Once the pin 336 reaches the axial section 342, the upper section 35 advances within the container 1. This sliding motion causes a protrusion, spike 346 to pierce the membrane 334 and so allows the lysis fluid 3 into lower chamber 348. A second projection 350 pierces the membrane at a position opposite a holding location 318. The holding location 318 encloses one or more beads 320 on all sides, apart from an opening 322 which faces the membrane 334.

Prior to rotation of the base section 302, the membrane 334 serves to retain the one or more beads 320 within the holding location 318.

Further rotation of the upper section 35 relative to the container 1 is controlled by the cooperation of the pin 336 and track 338. This rotation moves the second projection 350 out of alignment with the holding location 318 and provides the room for the one or more beads 320 to fall into the lower section 348 of the container 1.

As a result of these steps, FIG. 11b, the DNA on the swab 19, the lysis fluid 3 and the one or more beads 320 are all in contact with one another within the lower section 348 of the first container. This allows for the DNA to be released from the swab 19, lysed by the lysis fluid 3 and then become fixed to the one or more beads 320, for instance by virtue of the surface chemistry of the one or more beads 320 compared with that of the lysed DNA.

The lysis fluid and the DNA contained within it can be dispensed from the container 1 in a similar manner to that described above for Embodiment I.

### EMBODIMENT K

This embodiment provides a similar arrangement to the Embodiment I form. In this case, however, the holding location 318 for the one or more beads 320 is different and the storage location 360 for the lysis fluid 3 is different.

The lysis fluid 3 is held in a storage location 360 mounted on the side of the container 1. The storage location 360 is compressed by the user, FIG. 12d, to release the fluid 3 into the container 1.

The one or more beads are in a holding location 318 mounted on the side of the container 1. The one or more beads 320 are released into the lysis fluid 3, as shown in FIG. 12d by compressing the holding location 318 and causing the barrier between the holding location 318 and the container 1 to burst. This releases the one or more beads 320 into the lysis fluid 3. held

Again, the DNA, lysis fluid 3 and one or more beads 320 are provided in the same location as a result.

The dispensing of the lysis fluid 3 into the device 29 is achieved in an equivalent manner to that of Embodiment I.

### EMBODIMENT L

This embodiment's operation is shown in overview in FIG. 13 as the steps advance. The approach uses an embodiment in which the swab 19 is inserted through the upper section 35 to a position within the container 1.

Thus in Stage 1, the upper section 35 is sealed by a lid 9 and hence the container 1 is also sealed.

In Stage 2, the lid 9 is removed to allows insertion of the swab 19.

In Stage 3, the swab 19 is pushed through the upper section 35 and into the container 1. Here the swab 19 deforms a series of projections which then retain the swab 19 when the application 310 is removed. During its passage into container 1, the swab 19 breaks a seal on the lysis fluid store location 400 and breaks a lower seal on the lysis fluid store location 400, such that the lysis fluid 3 falls into the container 1.

By Stage 4, the swab 19 and lysis fluid 3 are in the container 1. The lid 9 is then replaced.

After the lysis process has been advanced, or even before, then in Stage 5, the upper section 35 is rotated relative to the container 1 and this frees the upper section 35 to advance into the container 1 to a controlled extent. This causes the seals on the holding location(s) 318 for one or more beads 320 to be broken and releases the one or more beads into the lysis fluid 3.

The base 402 of the container 1 is then pushed onto the device. This axial advancement of the container 1 on to the device causes the lower seal 404 to be broken and so form a fluid connection between the container 1 and the device, Stage 6.

In Stage 7, the upper section 35 is rotated relative to the container 1 and as a result, the upper section 35 can be moved from its expanded state and so drive the lysis fluid 3 into the device, Stage 8.

### EMBODIMENT M

A further embodiment is shown in FIGS. 14a to 14j. In this case, the upper section 35 again has an expanded state and the ability to move within the container 1.

In the illustrated sequence, the swab 19 has already been positioned and the lid 9 has been inserted to completely seal the swab 19 inside the container 1. The container 1 includes the lysis fluid 3 in contact with the swab 19.

The lid 9 is pushed down into the upper section 35 until a wider diameter ring section 410 on the lid 9 abuts the mouth section 332 and prevents further inward movement.

Rotation of the upper section 35 relative to the container 1, FIG. 14b, causes pin 336 to move along track 338. The track 338 has an initial circumferential section 340. The rotation causes the alignment of a protrusion, spike 346 with the membrane 334 and of a second projection 350 opposite a holding location 318. The holding location 318 encloses one or more beads 320 on all sides, apart from an opening 322 which faces the membrane 334.

After this rotation, FIG. 14c, an axial section 342 of the track 338 allows the upper section 35 to be advanced axial within the container 1. This sliding motion causes the protrusion, spike 346 to pierce the membrane 334 and so allows the lysis fluid 3 into lower chamber 348. The second projection 350 pierces the membrane at a position opposite a holding location 318.

A further circumferential section 344 of the track 338, FIG. 14d, allows further rotation of the upper section 35 relative to the container 1. This rotation moves the second projection 350 out of alignment with the holding location 318 and provides the room for the one or more beads 320 to fall into the lower section 348 of the container 1. During rotation, the projection tears open the membrane 334 it has pierced. The track 338 includes a stop 412 which resists any rotation of the upper section 35 in the reverse direction by engaging the pin 336.

As a result of these steps, FIG. 14d, the DNA on the swab 19, the lysis fluid 3 and the one or more beads 320 are all in contact with one another within the lower section 348 of the first container. This allows for the DNA to be released from the swab 19, lysed by the lysis fluid 3 and then become fixed to the one or more beads 320, for instance by virtue of the surface chemistry of the one or more beads 320 compared with that of the lysed DNA.

To assist the lysis, the container 1, and particularly the lower section 348 is moved to, FIG. 14e, and inserted into, FIG. 14f, a heating block 414. The heating block 414 includes a recess 416 designed to snugly receive the container 1. The container 1 is inserted until the bottom of the container 1 abuts the bottom of the recess 416. The temperature and/or other conditions applied to the contents of the container 1 whilst in this recess 416 are intended to assist the desired reactions and processes.

Once the lysis and/or other reactions have been completed to the desired degree, the container 1 is removed from the heating block 414 and moved to the cartridge 418 in which subsequent reactions and processes will be performed. As shown in FIG. 14g, the opening 420 into the cartridge 418 has a foil seal 422. This can either be removed or can be pierced by positioning the container 1 on the opening 420 to the cartridge 418. As shown, a screw thread 424 is provided externally on the mount on the cartridge 418.

As the container 1 is screwed onto the cartridge 418, the membrane 426 which seals the bottom of the container 1 is brought closer and closer to a protrusion, spike 428, provided inside the opening 420. The spike 428 eventually pierces the membrane 426 and provides the fluid connection into the cartridge 418, FIG. 14i.

The lysis fluid 3 and the DNA contained within it can be dispensed from the container 1 as shown in FIG. 14j. Downward pressure is applied by the user of a sufficient level to force the lid 9, which is in the form of a plunger, down into the upper section 35 despite the wider diameter ring section 410 on the lid 9 abutting the mouth section 332. The force is sufficient to cause enough deformation in the lid 9, ring section 410 and upper section 35 to allow axial movement. The axial movement increases the pressure within the lower section 348 and hence forces the fluid through into the opening 420 in the cartridge 418.

### EMBODIMENT N

In this embodiment, two different capacity containers are shown, FIG. 15, in exploded perspective view.

In each case, the container 1 is provided with a base section 500 which includes a lower central opening. A membrane 502 is provided to seal the opening and as a result the bottom of the lower section 348 defined by the lower section 348 when it is in cooperation with the base section 500.

The upper section 35 is also provided with a membrane 504 to seal its bottom.

A seal 506, for instance in the of membrane with a slit in it is provided within the upper section 35.

Finally, the lid 9, in plunger form, is provided.

O-rings 510a, 510b and 510c are provided to promote a good seal between the surfaces which slide relative to one another at various times in the operation of the cartridge 1.

As shown, the base section 500 is capable of cooperating with a pipette style tip 512 to allow dispense of the fluid from the container 1 into a location of choice. This can assist in certain cases with the easy of transferring liquid from the container to another location, for instance when testing the container system.

FIG. 16a shows a detailed cross-sectional perspective view of the container 1 and upper section 35. The mouth 332 of the upper section 35 is shown with the swab applicator 310 and swab 19 inserted. The swab 19 is in the position where it will be retained.

The retention position is within a cylindrical element 600. A series of radial webs 602 mount the cylindrical element 600 relative to the upper section 35. The annular upper surface 604 and webs 602 provide a support surface for the slit seal 606, shown in FIG. 16c, but absent from FIG. 16a for clarity purposes. At its lower end, a continuous web 608 is provided. This reduces the overall volume of the lower section 348 so as to assist in ensuring the plunger, when activated is able to fully dispense the lysis fluid 3. The lower section 348 is provided with an inclined surface 610 which leads down to the central opening 612 which is closed by membrane 502.

The underside of the upper section 35 is seen in FIG. 16b. The membrane 504 which is supported by annulus 614 and ribs 616 and partial annulus 618 is removed in this figure for the purposes of clarity. The central cylindrical section 600 and continuous web 608 can be seen. The annulus 614, ribs 616, partial annulus 618 and continuous web 608 define the perimeters of two holding locations 620 and 622. These holding locations are covered by the membrane 504 so as to seal them completely before use.

One of the holding locations 620 contains the lysis fluid 3. The other holding location 622 contains one or more beads. When the swab 19 is pushed down through the membrane 504, this releases the lysis fluid 3 and beads into contact with the swab 19. This configuration can be seen in FIG. 16c.

FIG. 16d shows the pipette style tip 512 in place and the fluid flow route down into the tip.

1. A container, the container including an opening to a chamber, closure means for the opening and discharge means for the container.

2. The container of claim 1 in which the container is provided with a plurality of chambers, one or more of the chambers being physically isolated from one or more of the other chambers, with one or more of the other chambers being providing with one or more reagents.

3. The container of claim 2 in which the one or more other chambers are separated from the one or more chambers by a seal or a plurality of seals.

4. The container of claim 2 in which the container includes means to break the seal between one or more of the chambers and one or more of the other chambers.

5. The container of claim 4 in which relative movement of the one or more chambers compared with the one or more other chambers breaks the seal between them.

6. The container of claim 2 in which one of the one or more chambers has an axis and the one or more other chambers are off set from the axis.

7. The container of claim 1 in which one or more further chambers are providing with one or more sample processing aids.

8. The container of claim 1 in which the container is provided with a chamber in to which the sample is introduced and another chamber in which the reagents are present and/or a further chamber in which one or more sample processing aids are present within the further chamber.

9. The container of claim 1 in which the container is provided with a first chamber which is accessed through an opening and the container is provided with a second chamber which is accessed through the first chamber, the second chamber being provided with one or more further chambers and/or one or more other chambers.

10. The container of claim 9 in which the one or more further chambers and/or one or more other chambers are provided leading into the second chamber.

11. The container of claim 9 in which the one or more further chamber and/or one or more other chambers are provided in the base of the first chamber.

12. The container of claim 9 in which the container includes a first section which is rotatable relative to a second section.

13. The container of claim 9 in which the container includes a first chamber defining section and a second chamber defining section, the first chamber defining section being at least partially received within the second chamber defining section.

14. The container of claim 13 in which the first chamber defining section provides a pre-first chamber defining section.

15. The container of claim 13 in which the first chamber defining section may be rotatable relative to the second chamber defining section.

16. The container of claim 13 in which one or more stops or other locator means are provided to control the position of the first chamber defining section relative to the second chamber defining section.

17. The container of claim 16 in which the locator means are confined by a track.

18. The container of claim 17 in which the track includes a first perimeter section extending circumferentially and the track includes one or more further perimeter extending sections.

19. The container of claim 17 in which the track includes one or more axially extending sections and one perimeter extending section is joined to another by an axially extending section.

20. The container of claim 17 in which the container has a pre-use state and the container includes one or more reagents in the pre-use state.

21. The container of claim 17 in which the container has a use state and one or more chambers within the container are accessible in the use state to a sample collection device.

22. The container of claim 17 in which the container has a sample to fluid contacting state in which one or more fluids are released into one or more chambers containing the sample.

23. The container of claim 17 in which the container has a sample to sample processing aids contacting state in which one or more sample processing aids are released into one or more chambers containing the sample or vice versa.

24. The container of claim 17 in which the container has an initial processing state in which the container and/or its contents are heated and/or agitated.

25. The container of claim 17 in which the container has a transfer state in which at least a part of the contents of the container are transferred to a device.

26. The container of claim 17 in which the container has an expanded state and a compressed state and fluid passes from the container to the device during the transition between expanded state to compressed state and/or whilst in the compressed state.

27. The container of claim 17 in which the container is provided with an internal heat source.

28. The container of claim 17 in which the container contains one or more sample processing aids, used to assist and/or to provide a separation between one or more components of the sample and one or more other components.

29. The container of claim 17 in which the container contains one or more reagents or material for amplification.

30. The container of claim 17 in which the container contains one or more reagents or materials for use in a size based separation and/or for use in electrophoresis

31. A method of preparing a sample, the method including:
collecting the sample;
introducing the sample to a container;
one or more preparing steps being applied to the sample whilst in the container.

32. The method of claim 31 in which the container has a pre-use state and the container
includes one or more reagents in the pre-use state.

33. The method of claim 31 in which the container has a use state and one or more chambers within the container are accessible in the use state to a sample collection device.

34. The method of claim 31 in which the container has a sample to fluid contacting state in which one or more fluids are released into one or more chambers containing the sample.

35. The method of claim 31 in which the container has a sample to sample processing aids contacting state in which one or more sample processing aids are released into one or more chambers containing the sample or vice versa.

36. The method of claim 31 in which the container has an initial processing state in which the container and/or its contents are heated and/or agitated.

37. The method of claim 31 in which the container has a transfer state in which at least a part of the contents of the container are transferred to a device.

38. The method of claim 31 in which the container has an expanded state and a compressed state and fluid passes from the container to the device during the transition between expanded state to compressed state and/or whilst in the compressed state.
